(19) United States
(12) Patent Application Publication
(10) Pub. No.: US 2026/0033725 A1
(43) Pub. Date: Feb. 5, 2026

(54) PHOTOACOUSTIC IMAGING METHODS AND PHOTOACOUSTIC IMAGING SYSTEMS

(71) Applicants: SHENZHEN MINDRAY BIO-MEDICAL ELECTRONICS CO., LTD., Shenzhen (CN); BeiJing Shen Mindray Medical Electronics Technology Research Institute Co., Ltd., Beijing (CN)

(72) Inventors: Yuchao SANG, Shenzhen (CN); Fang YANG, Shenzhen (CN); Maodong SANG, Shenzhen (CN); Lei ZHU, Shenzhen (CN)

(73) Assignees: SHENZHEN MINDRAY BIO-MEDICAL ELECTRONICS CO., LTD., Shenzhen (CN); BeiJing Shen Mindray Medical Electronics Technology Research Institute Co., Ltd., Beijing (CN)

(21) Appl. No.: 19/287,803

(22) Filed: Jul. 31, 2025

(30) Foreign Application Priority Data
Aug. 1, 2024 (CN) 202411054845.9

(51) Int. Cl. A61B 5/00; A61B 5/107; A61B 5/145

(52) U.S. Cl. CPC A61B 5/0095; A61B 5/1075; A61B 5/14542; A61B 5/7203; A61B 5/7235; A61B 5/7271; A61B 5/7425

(57) ABSTRACT

Disclosed are a photoacoustic imaging method and system. The method comprises: transmitting ultrasound to target tissue and receiving echoes to obtain an ultrasound echo signal; generating an ultrasound image from the echo signal; transmitting first and second laser pulses (each having a corresponding wavelength) to the tissue and receiving corresponding first and second photoacoustic signals; obtaining a blood oxygen saturation (SaO2) signal based on the first and second photoacoustic signals to generate a SaO2 image; determining a target region and a reference region in the SaO2 image; obtaining a first confidence level of the SaO2 signal in the target region; and displaying the SaO2 image, the ultrasound image and the first confidence level on a display interface. The present disclosure can improve the reliability and effectiveness of displayed SaO2 results.

### TECHNICAL FIELD

The present disclosure relates to the technical field of ultrasound imaging, and more particularly to photoacoustic imaging methods and photoacoustic imaging systems.

### BACKGROUND

Photoacoustic tomography (PAT) is a non-invasive medical imaging technical that combines the high contrast advantage of optical imaging with the deep penetration advantage of ultrasound imaging. This technical has applications in clinical diagnosis and functional imaging of tissue. The photoacoustic effect, the underlying principle of PAT, involves irradiation of biological tissue with a pulsed laser. The tissue absorbs electromagnetic energy, causing thermal expansion. This rapid thermal expansion generates acoustic pressure waves that propagate outward through the tissue as mechanical waves. These generated acoustic pressure waves are detected by an ultrasound transducer. The detected signals are then processed by reconstruction algorithms to reconstruct the initial acoustic pressure field at the time of the pulsed laser excitation. This reconstructed pressure field corresponds to an image (or map) representing the optical absorption distribution within the tissue. By detecting ultrasound waves via a broadband ultrasound transducer rather than scattered photons as in optical imaging, PAT inherently avoids interference from optical scattering. This enables PAT to provide tissue images (or reconstructions) with high contrast and high spatial resolution. Photoacoustic signals depend on both the optical and acoustic properties of biological tissues. Differences in optical absorption by tissue under various physiological conditions correspond to variations in tissue properties, including its structure, metabolism, pathology, and neural activity.

The intensity of the ultrasound signal generated by the photoacoustic effect depends on the delivered laser energy, the optical parameters of the tissue, and the coupling efficiency at the transmission interface. To ensure operational safety during photoacoustic imaging process, the laser energy is constrained within a defined range to prevent tissue damage or adverse biological effects. However, this constraint limits the achievable imaging depth and signal intensity in photoacoustic imaging. Specifically when detecting weak acoustic signals originating from depths exceeding 2 cm, the signal intensity approaches the noise floor. Notably, methods for detecting blood oxygen saturation via photoacoustic imaging is highly sensitive to noise. Computations performed using ultrasound signal data characterized by a low signal-to-noise ratio (SNR) yields results that are subject to fluctuation and significant errors, thereby failing to accurately represent the true blood oxygen saturation value at the measurement location. Furthermore, in dual-wavelength implementations, variations in both the delivered laser energy and the tissue absorption coefficient between the two different wavelengths results in corresponding variations in the SNR of the acquired ultrasound signals at each wavelength. This SNR disparity further compounds the overall measurement error. Additionally, practical limitations imposed by equipment specifications and operational degradation cause the energy and wavelength output by the laser source generating the photoacoustic signals to deviate from their setpoint values. These output deviations contribute to inaccuracies in the computed blood oxygen saturation values. The depth location and spatial orientation of the interrogated vasculature constitute further factors influencing the blood oxygen saturation results. The utilization of the blood oxygen saturation calculation functionality is compromised when such inaccuracies or anomalous results occur, significantly impairing user interpretation and diagnostic assessment.

### SUMMARY

The summary of the Disclosure introduces a series of concepts in simplified form, which are described in further detail in the Detailed Description section. The Summary is not intended to identify key features or essential technical features of the claimed invention, nor should it be used construe the scope of the claimed subject matter.

According to an aspect of the present disclosure, a photoacoustic imaging method disclosed in some embodiments may include: controlling a transducer of a photoacoustic imaging probe to transmit ultrasound waves to a target tissue and receive echoes of the ultrasound waves to obtain an ultrasound echo signal; generating an ultrasound image based on the ultrasound echo signal; controlling a laser of the photoacoustic imaging probe to transmit a first laser pulse of a first wavelength and a second laser pulse of a second wavelength to the target tissue, and controlling the transducer of the photoacoustic imaging probe to receive a first photoacoustic signal corresponding to the first laser pulse and a second photoacoustic signal corresponding to the second laser pulse that are returned from the target tissue; obtaining a blood oxygen saturation signal based on the first photoacoustic signal and the second photoacoustic signal, and generating a blood oxygen saturation image based on the blood oxygen saturation signal; determining a target region and a reference region in the blood oxygen saturation image; determining a first target SNR of the first photoacoustic signal corresponding to the target region and a first reference SNR of the first photoacoustic signal corresponding to the reference region; determining a second target SNR of the second photoacoustic signal corresponding to the target region and a second reference SNR of the second photoacoustic signal corresponding to the reference area; obtaining a first confidence level of the blood oxygen saturation signal in the target region based on the first target SNR, the first reference SNR, the second target SNR and the second reference SNR; and displaying the blood oxygen saturation image, the ultrasound image and the first confidence level on a display interface.

According to another aspect of the present disclosure, a photoacoustic imaging method disclosed in some embodiments may include: controlling a photoacoustic probe to transmit ultrasound waves to a target tissue and receive echoes of the ultrasound waves to obtain an ultrasound echo signal, and generating an ultrasound image based on the echo signal; transmitting a laser pulse to the target tissue and controlling the photoacoustic probe to receive a photoacoustic signal returned from the target tissue; generating a photoacoustic image based on the photoacoustic signal; determining a target region and a reference region in the photoacoustic image; determining a target SNR of the photoacoustic signal corresponding to the target region and a reference SNR of the photoacoustic signal corresponding to the reference region; obtaining the confidence level of the photoacoustic signal of the target region based on the target SNR and the reference SNR; and displaying the ultrasound image, the photoacoustic image, and the confidence level on a display interface.

According to aspect of the present disclosure, a photoacoustic imaging method disclosed in some embodiments may includes: controlling a photoacoustic probe to transmit ultrasound waves to a target tissue and receive echoes of the ultrasound waves to obtain an ultrasound echo signal, and generating an ultrasound image based on the echo signal; transmitting a first laser pulse and a second laser pulse to the target tissue, and controlling the photoacoustic probe to receive a first photoacoustic signal corresponding to the first laser pulse and a second photoacoustic signal corresponding to the second laser pulse that are returned from the target tissue; determining a first emission energy and a first wavelength of the first laser pulse, as well as a second emission energy and a second wavelength of the second laser pulse; obtaining a blood oxygen saturation signal based on the first photoacoustic signal and the second photoacoustic signal, and generating a blood oxygen saturation image based on the blood oxygen saturation signal; obtaining pre-established correspondence relationships between different wavelengths, different emission energies and blood oxygen saturation deviations; determining a blood oxygen saturation deviation corresponding to the first emission energy, the first wavelength, the second emission energy and the second wavelength based on the correspondence relationships, and determining the confidence level of the blood oxygen saturation signal based on the blood oxygen saturation deviation; and displaying the blood oxygen saturation image, the ultrasound image and the confidence level on a display interface.

According to another aspect of the present disclosure, a photoacoustic imaging method disclosed in some embodiments may include: controlling a photoacoustic probe to transmit ultrasound waves to a target tissue and receive echoes of the ultrasound waves to obtain an ultrasound echo signal, and generating an ultrasound image based on the echo signal; transmitting a first laser pulse and a second laser pulse to the target tissue, and controlling the photoacoustic probe to receive a first photoacoustic signal corresponding to the first laser pulse and a second photoacoustic signal corresponding to the second laser pulse that are returned from the target tissue; obtaining a blood oxygen saturation signal based on the first photoacoustic signal and the second photoacoustic signal, and generating a blood oxygen saturation image based on the blood oxygen saturation signal; determining a target region in the blood oxygen saturation image; determining a vascular depth and a vascular orientation in the target region; obtaining pre-established correspondence relationships between different vascular depths, different vascular orientations and blood oxygen saturation deviations; determining a blood oxygen saturation deviation corresponding to said vascular depth and said vascular direction based on the correspondence relationships, and determining a confidence level of the blood oxygen saturation signal in the target region based on said blood oxygen saturation deviation; and displaying the blood oxygen saturation image, the ultrasound image and the confidence level in a display interface.

According to another aspect of the present disclosure, a photoacoustic imaging method in some embodiments may include: controlling a photoacoustic probe to transmit ultrasound waves to a target tissue and receive echoes of the ultrasound waves to obtain an ultrasound echo signal, and generating an ultrasound image based on the echo signal; transmitting a first laser pulse and a second laser pulse to the target tissue, and controlling the photoacoustic probe to receive a first photoacoustic signal corresponding to the first laser pulse and a second photoacoustic signal corresponding to the second laser pulse that are returned from the target tissue; obtaining a blood oxygen saturation signal based on the first photoacoustic signal and the second photoacoustic signal, and generating a blood oxygen saturation image based on the blood oxygen saturation signal; determining a target region in the blood oxygen saturation image; determining a confidence level of the blood oxygen saturation signal in the target region; and displaying the blood oxygen saturation image, the ultrasound image and the confidence level on a display interface.

According to another aspect of the present disclosure, a photoacoustic imaging system in some embodiments may include: a photoacoustic imaging probe, a processor and a display; wherein the photoacoustic imaging probe includes a transducer and a laser and is used to transmit ultrasound waves to a target tissue and receive echoes of the ultrasound waves via the transducer to obtain an ultrasound echo signal; and transmit a first laser pulse of a first wavelength and a second laser pulse of a second wavelength to the target tissue via the laser, and receive a first photoacoustic signal corresponding to the first laser pulse and a second photoacoustic signal corresponding to the second laser pulse that are returned from the target tissue via the transducer; the processor is used to: generate an ultrasound image based on the ultrasound echo signal; obtain a blood oxygen saturation signal based on the first photoacoustic signal and the second photoacoustic signal, and generate a blood oxygen saturation image based on the blood oxygen saturation signal; determine a target region and a reference region in the blood oxygen saturation image; determine a first target SNR of the first photoacoustic signal corresponding to the target region and a first reference SNR of the first photoacoustic signal corresponding to the reference region; determine a second target SNR of the second photoacoustic signal corresponding to the target region and a second reference SNR of the second photoacoustic signal corresponding to the reference region; and obtain a first confidence level of the blood oxygen saturation signal of the target region based on the first target SNR, the first reference SNR, the second target SNR and the second reference SNR; and the display is used to display the blood oxygen saturation image, the ultrasound image and the first confidence level.

The photoacoustic imaging methods and systems according to embodiments of the present disclosure process photoacoustic signals generated by laser excitation at two different wavelengths. Based on a target SNR and a reference SNR, the methods and systems obtain a confidence level of a blood oxygen saturation signal in a target region, thereby enabling users to evaluate reliability and validity of displayed blood oxygen saturation results. The system concurrently displays on a display interface: a blood oxygen saturation image, an ultrasound image, and the confidence level, facilitating user analysis and processing of the displayed results.

### DESCRIPTION OF THE DRAWINGS

The above and other aspects, features and advantages of the present disclosure will become more apparent from the following detailed description of embodiments when taken in conjunction with the accompanying drawings. The accompanying drawings are provided to further illustrate the embodiments and form part of the specification. Together with the detailed description, the drawings serve to explain the principles of the invention without limiting its scope. In the drawings, identical reference characters designate corresponding components or steps throughout the figures.

FIG. 1 illustrates a block diagram of a photoacoustic imaging system in accordance with one or more embodiments of the present disclosure;

FIG. 2 illustrates a schematic flowchart of a photoacoustic imaging method according to a first embodiment of the present disclosure;

FIG. 3 illustrates selection of a target region and a reference region in accordance with one or more embodiments of the present disclosure;

FIG. 4 illustrates blood oxygen saturation levels without noise suppression and with noise suppression in accordance with one or more embodiments of the present disclosure;

FIG. 5 illustrates comparison of photoacoustic signals before and after noise filtering according to one or more embodiments of the present disclosure;

FIG. 6(a) illustrates display of confidence levels in accordance with one or more embodiments of the present disclosure;

FIG. 6(b) illustrates display of confidence levels in accordance with one or more embodiments of the present disclosure;

FIG. 7 illustrates a schematic flowchart of a photoacoustic imaging method in accordance with a second embodiment of the present disclosure;

FIG. 8 illustrates a schematic flowchart of a photoacoustic imaging method in accordance with a third embodiment of the present disclosure;

FIG. 9 illustrates a laser optical path output in accordance with one or more embodiments of the present disclosure;

FIG. 10 illustrates a COMSOL breast model in accordance with one or more embodiments of the present disclosure;

FIG. 11 illustrates a schematic flowchart of a photoacoustic imaging method in accordance with a fourth embodiment of the present disclosure;

FIG. 12(a) illustrates light flux distribution on an imaging plane in accordance with one or more embodiments of the present disclosure;

FIG. 12(b) illustrates light flux distribution along depth at a central position in accordance with one or more embodiments of the present disclosure; and

FIG. 13 illustrates a schematic flowchart of a photoacoustic imaging method in accordance with a fifth embodiment of the present disclosure.

### DETAILED DESCRIPTION

To make the objectives, technical solutions and advantages of the present invention more apparent, exemplary embodiments will be described in detail below with reference to the accompanying drawings. It should be understood that the described embodiments represent only some examples of the present invention, and are not intended to be all possible embodiments. The present invention is not limited to the exemplary embodiments described herein. Other embodiments that could be made by those skilled in the art based on the teachings herein would fall within the intended scope of the invention.

In the following description, specific details are provided to promote a fuller understanding of the present invention. It is will be apparent, however, to those skilled in the art that the invention may be practices without one or more of these details. In other instances, well-known technical features have been omitted for brevity to avoid obscuring the described embodiments of the invention.

It should be understood that the invention may be practice in various forms and is not be construed as limited to the embodiments set forth herein. Rather, these embodiments are provided so that this disclosure will be full and enabling, and will clearly convey the essential scope of the invention as defined by the claims to those skilled in the art.

The terminology used herein is for the purpose of describing particular embodiments only and is not to be construed as limiting the invention. As used herein, singular references (“a”, “an” and “the”) are intended to include the plural forms as well, unless the context clearly indicates otherwise. It is further to be understood that the terms “comprise” and/or “include”, when used in this specification, specify the presence of the stated features, integers, steps, operations, elements and/or components, but do not preclude the presence or addition of one or more other features, integers, steps, operations, elements, components and/or groups. As used herein, the term “and/or” includes any and all combinations of one or more of the associated listed items.

To facilitate an understanding of the invention, detailed embodiments will be described below setting forth the structure and steps of the invention. Detailed descriptions of exemplary embodiments are provided in the following sections. It is to be understood, however, that the invention is not limited to the specific embodiments described herein.

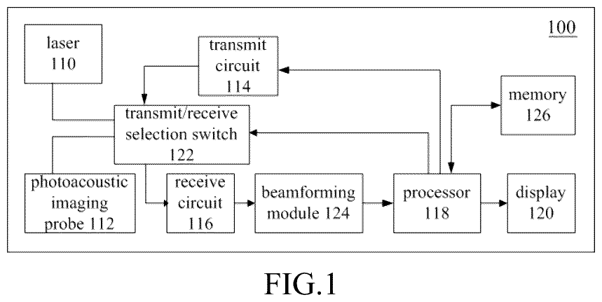

Below, first, with reference to FIG. 1, a description is provided of a photoacoustic imaging system according to an embodiment of the present disclosure. FIG. 1 shows a schematic block diagram of a photoacoustic imaging system 100 according to an embodiment of the present invention.

As shown in FIG. 1, the photoacoustic imaging system 100 comprises a laser 110, a photoacoustic imaging probe 112, a transmit circuit 114, a receive circuit 116, a processor 118 and a display 120. Further, the photoacoustic imaging system may also include a transmit/receive selection switch 122 and a beamforming module 124. The transmit circuit 114 and the receive circuit 116 may be connected to the photoacoustic imaging probe 112 through the transmit/receive selection switch 122. The laser 110 may be included inside the photoacoustic imaging probe 112.

The laser 110 is configured to transmit laser to an imaging target. After transmitting laser to the imaging target, the receive circuit 116 can receive a photoacoustic signal returned from the imaging target under the excitation of the laser through the photoacoustic imaging probe 112. This photoacoustic signal is directly or after being processed sent to the processor 118, which processes the photoacoustic signal to obtain photoacoustic image data of the imaging target.

In one embodiment of the present application, the laser 110 is operatively coupled to the transmit/receive selection switch 122 configured to control laser emission. The laser 110 may also be optically connected to the photoacoustic imaging probe 112 via an optical transmission conduit. An optical fiber bundle is coupled to the photoacoustic imaging probe 112, wherein the optical fiber bundle conducts the laser radiation to both lateral sides of the photoacoustic imaging probe 112 to deliver back-illumination to the imaging target. In some implementations, both the laser 110 and the optical fiber bundle are internally housed within the photoacoustic imaging probe 112. The photoacoustic imaging probe 112 further includes a plurality of ultrasound transducer elements for ultrasound imaging, such that the photoacoustic imaging probe 112 is adapted for both conventional ultrasound imaging and photoacoustic imaging.

The plurality of transducer elements within the photoacoustic imaging probe 112 define a two-dimensional array configuration, and are alternatively configurable as a convex array. Each transducer element is configured to: (a) transmit ultrasound waves in response to an excitation electrical signal, and (b) convert the received ultrasound waves into corresponding electrical signals, thereby establishing bidirectional energy conversion between electrical pulse signals and ultrasound waves. The array is thus operable to: (i) direct ultrasound waves toward the tissue of the imaging target, and (ii) receive the ultrasound wave echoes reflected by the tissue.

During ultrasound imaging, a transmit sequence and a receive sequence control: (a) which transducer elements operate in a transmit mode to transmit ultrasound waves, (b) which transducer elements operate in a receive mode to detect ultrasound echoes, or (c) time-division multiplexing of the transducer elements between transmit and receive mode. The transducer elements engaged in ultrasound transmission are excitable by: (i) synchronous electrical signals to generate simultaneous ultrasound wave emission; or, (ii) staggered electrical signals having predetermined time intervals to produce successive ultrasound wave emissions separated by corresponding time intervals.

During ultrasound imaging, the transmit circuit 114, operably coupled to the processor 118, generates a transmit sequence. The transmit sequence control some or all of the transducer elements to emit ultrasound waves toward the target tissue. The transmit sequence parameters include: (a) spatial distribution parameters defining a number and location of transducer elements utilized for the emission, and (b) acoustic beam parameters, including but not limited to amplitude, frequency, pulse repetition interval, emission angle, waveform, and focal position. In some implementations, the transmit circuit 114 further applies a phase delay profile to the beam formation. Applying the phase delay profile causes different transducer elements to emit the ultrasound waves at different times, so that the emitted ultrasound beams focus at a predetermined region of interest. The transmit sequence parameters can differ for different imaging modes. Ultrasound echo signals received by the receive circuit 116 are processed by downstream signal processing circuitry using algorithms corresponding to the respective imaging mode, to thereby generate ultrasound images specific to the respective imaging mode.

The receive circuit 116 may include one or more amplifiers, and one or more analog-to-digital converters. The one or more amplifiers amplify the received ultrasound echo signal or photoacoustic signal after application of appropriate gain compensation. The one or more analog-to-digital converters sample the (amplified) signal at predetermined time intervals, thereby converting it into a digital signal. The digitized signal retains characteristic information comprising amplitude, frequency, and phase. The receive circuit 116 provides the digitized signal to the beamforming module 124 for processing.

The beamforming module 124 performs processing operations-including focusing delay, weighting, and channel summation-on the ultrasound echo signals or photoacoustic signals. The processed signals are transmitted to the processor 118. The processor 118 executes signal processing operations comprising signal detection, signal enhancement, data conversion, and logarithmic compression to generate ultrasound images or photoacoustic images. The ultrasound images or photoacoustic images produced by the processor 118 are configured to be displayed on the display 120, or stored in a memory 126.

Optionally, the processor 118 may be implemented as software, hardware, firmware, or any combination thereof. The processor 118 comprises, or is implemented as: a single or multiple application-specific integrated circuits (ASICs), a single or multiple general-purpose integrated circuits, a single or multiple microprocessors, a single or multiple programmable logic devices, or any combination thereof. Other suitable circuits or devices may also be used. Moreover, the processor 118 is configured to control other components in the photoacoustic imaging system 100 to perform corresponding steps of the methods in the various embodiments described in this specification.

The display 120 is connected to the processor 118. The display 120 may be, for example, a touch screen, a liquid crystal display (LCD), or an LCD monitor or television separate from the photoacoustic imaging system 100. Alternatively, the display 120 may be an electronic device such as a smart phone or a tablet computer, etc. The display 120 may be singular or plural.

The display 120 may show the ultrasound or photoacoustic images produced by the processor 118. Additionally, while displaying the ultrasound or photoacoustic images, the display 120 may also provide a graphical interface for human-computer interaction to users. One or more controllable objects may be provided on the graphical interface, allowing users to input operation instructions through a human-computer interaction device to control these controllable objects and thereby perform corresponding control operations. For instance, an icon may be displayed on the graphical interface, and a user may operate on the icon through a human-computer interaction device to execute specific functions, such as drawing a region of interest box on the ultrasound image.

Optionally, the photoacoustic imaging system 100 may include an additional human-machine interaction (HMI) device besides the display 120. This additional HMI device is connected to the processor 118. For example, the processor 118 may be connected to the HMI device via an external input/output (I/O) port. The external I/O port may be a wireless transceiver, a wired transceiver, or a combination thereof. In addition, the external I/O port may be configured to communicate using USB, at least one bus protocol (e.g., CAN), and/or at least one wired network protocol.

The HMI device may include an input device configured to detect user input information. By way of examples, the user input information may correspond to: control instructions for the timing of ultrasound transmission or reception; operation input instructions for drawing graphical elements including a point, a line, or a box on an ultrasound image or a photoacoustic image, or a further instruction type. The input device may include, by way of examples and not limitation, one or more of: a keyboard, a mouse, a scroll wheel, a trackball, a mobile input device (such as a mobile device with a touch screen, or a mobile phone), and a multifunctional control knob. The HMI device may further include an output device, such as a printer.

The photoacoustic imaging system 100 may also include a memory 126 configured to store: processor-executable instructions, received ultrasound echoes or photoacoustic signals, as well as ultrasound images or photoacoustic images, among other data. The memory may comprise a flash memory device, solid-state memory, a hard disk, or other storage media. The memory may include volatile memory and/or non-volatile memory, and may be removable or non-removable.

It should be understood that the components included in the photoacoustic imaging system 100 shown in FIG. 1 are merely illustrative, and the system may include more or fewer components. The present disclosure is not limited in this regard.

Oxygen saturation (SaO2) is a key physiological parameter pertaining to the respiratory and circulatory systems. SaO2 is defined as the percentage of oxyhemoglobin (HbO2) (hemoglobin bound to oxygen) relative to the total functional hemoglobin (Hb) (capable of binding oxygen) in the blood, providing a measure of the oxygen concentration in the blood. Clinically, arterial oxygen saturation (SaO2) is measured as an assessment of blood oxygen content. In healthy humans, normal arterial SaO2 is typically 98%, whereas venous oxygen saturation is approximately 75%. Different organs and tissues within the body exhibit varying metabolic rates and consequently have different blood flow requirements. A deficiency in oxygen supply directly impairs normal cellular metabolism, resulting in abnormal metabolic states within the hypoxic organs/tissues. Correspondingly, associated physiological structures and functional parameters are altered. For instance, cancerous tissue regions commonly exhibit a characteristic known as hypervascular hypoxia compared to normal tissues. (Hypervascular hypoxia is a condition marked by increased blood vessel density (hypervascularity) but insufficient oxygen supply (hypoxia)). Photoacoustic imaging utilizes a dual-wavelength tissue detection technique that exploit the differences in optical absorption characteristics between hemoglobin (Hb) and oxyhemoglobin (HbO2) at distinct spectral bands. Specifically, infrared light at two different, preselected wavelengths is directed onto the tissue under examination. The absorption of this light by Hb in the blood induces the photoacoustic effect, generating ultrasound signals with amplitudes corresponding to the absorption properties of Hb and HbO2 at their respective wavelengths. Detection of these differential amplitude signals allows for the quantification of oxygen saturation within the tissue.

Methods for detecting blood oxygen saturation (SaO2) using photoacoustic imaging exhibit high sensitive to noise. Calculation performed on ultrasound signal data with a low SNR yield results that exhibit excessive variability and significant errors, which fail to accurately represent the true SaO2 at the imaged location. Additionally, in dual-wavelength implementations, differential laser energy delivery and tissue absorption properties at the respective wavelengths lead to differing SNRs in the acquired ultrasound signals for each wavelength. This disparity thereby increases measurement error. Moreover, practical limitation arising from operational constraints and temporal degradation cause the energy output and wavelength of the laser source generating the photoacoustic excitation to vary from their nominal values. These instabilities introduce further inaccuracies into the calculated SaO2. Furthermore, the depth of the target vasculature and its orientation relative to the acoustic detector also influence the resulting SaO2 measurement. The presence of such artifacts (e.g., excessive noise, wavelength-dependent SNR differences, laser instabilities, depth/orientation effects) within the SaO2 calculation data can significantly degrade its reliability and complicate clinical interpretation by a user.

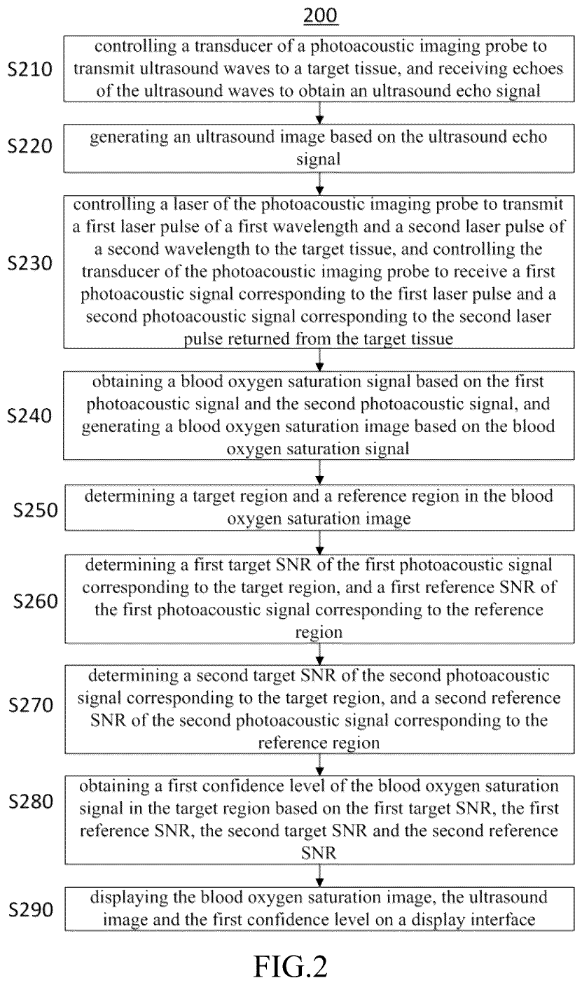

In view of the above problems, the present application discloses a photoacoustic imaging method. Hereinafter, the photoacoustic imaging method 200 according to an embodiment of the present application will be described with reference to FIG. 2. FIG. 2 is a schematic flowchart of the photoacoustic imaging method 200 according to an embodiment of the present application.

As shown in FIG. 2, the photoacoustic imaging method 200 in an embodiment of the present disclosure includes the following steps:
Step S210: controlling a transducer of a photoacoustic imaging probe to transmit ultrasound waves to a target tissue, and receiving echoes of the ultrasound waves to obtain an ultrasound echo signal;
Step S220: generating an ultrasound image based on the ultrasound echo signal;
Step S230: controlling a laser of the photoacoustic imaging probe to transmit a first laser pulse of a first wavelength and a second laser pulse of a second wavelength to the target tissue, and controlling the transducer of the photoacoustic imaging probe to receive a first photoacoustic signal corresponding to the first laser pulse and a second photoacoustic signal corresponding to the second laser pulse that are returned from the target tissue;
Step S240: obtaining a blood oxygen saturation signal based on the first photoacoustic signal and the second photoacoustic signal, and generating a blood oxygen saturation image based on the blood oxygen saturation signal;
Step S250: determining a target region and a reference region in the blood oxygen saturation image;
Step S260: determining a first target SNR of the first photoacoustic signal corresponding to the target region, and a first reference SNR of the first photoacoustic signal corresponding to the reference region;
Step S270: determining a second target SNR of the second photoacoustic signal corresponding to the target region, and a second reference SNR of the second photoacoustic signal corresponding to the reference region;
Step S280: obtaining a first confidence level of the blood oxygen saturation signal in the target region based on the first target SNR, the first reference SNR, the second target SNR and the second reference SNR; and
Step S290: displaying the blood oxygen saturation image, the ultrasound image and the first confidence level on a display interface.

The photoacoustic imaging method 200 according to an embodiment of the present disclosure processes photoacoustic signals generated by the laser excitation at two different wavelengths, and obtains a first confidence level for a blood oxygen saturation signal in a target region based on a target SNR and a reference SNR. This enables assessment of the reliability and validity of displayed results of blood oxygen saturation. Concurrently, a blood oxygen saturation image, an ultrasound image and the first confidence level are displayed on the display interface to facilitate user analysis and processing of the displayed results.

Furthermore, the photoacoustic imaging requires laser excitation at at least two different wavelengths of laser to obtain wavelength-specific photoacoustic image data. Accordingly, a first laser pulse having a first wavelength may be transmitted towards the imaging target, and a first photoacoustic signal corresponding to the first laser pulse may be received. Also, a second laser pulse having a second wavelength may be transmitted towards the imaging target, and a second photoacoustic signal corresponding to the second laser pulse may be received. Here, the first wavelength is different from the second wavelength, for example, the first laser may have a shorter wavelength relative to the longer wavelength of the second laser pulse. Specifically, upon user activation of a blood oxygen saturation calculation function during photoacoustic imaging, the system performs the following steps: (i) reading the operation instruction, (ii) verifying the status of a current imaging scan frame, (iii) initiating data acquisition upon commencement of the subsequent scan frame group; (iv) grouping the photoacoustic data uploads according to a predefined scan sequence to separate data corresponding to the first laser pulse from data corresponding to the second laser pulse; and (v) determining laser wavelength association by comparing matrix values of the uploaded data. Here, a photoacoustic data value associated with a shorter wavelength typically exceeds that of a longer wavelength, thereby enabling determination of whether the subsequent uploaded data corresponds to the first photoacoustic signal or the second photoacoustic signal.

In some embodiments, the determination of the first target SNR of the first photoacoustic signal corresponding to the target region, and the first reference SNR of the first photoacoustic signal corresponding to the reference region may include the following steps S2601 to S2605:
Step S2601: determining a first noise reference value based on the first photoacoustic signal;
As an example, the median of the first photoacoustic signal may be taken as the first noise reference value.

Step S2602: determining an average of the non-zero signal intensities in the first photoacoustic signal corresponding to the target region, so as to obtain a first effective signal intensity;
Step S2603: obtaining the first target SNR based on a ratio of the first effective signal intensity to the first noise reference value;

It should be noted that when there is no signal in the target region, the first target SNR is 0.

As an example, the first target SNR may be calculated by the following formula:

$$S N R 01 = db ( signal 01 / N )$$

where SNR01 represents the first target SNR, signal01 represents the first effective signal intensity, and N represents the first noise reference value.

Step S2604: determining an average of non-zero signal intensities in the first photoacoustic signal corresponding to the reference region to obtain the second effective signal intensity; and
Step S2605: obtaining the first reference SNR based on a ratio of the second effective signal intensity to the first noise reference value.

It should be noted that when there is no signal in the reference region, the first reference SNR is 0.

As an example, the first reference SNR may be calculated by the following formula:

$$SNR\_re1 = db ( signal 02 / N )$$

where SNR_re1 represents the first reference SNR, and signal02 represents the second effective signal intensity.

In some embodiments, the second target SNR and the second reference SNR may be determined by any suitable way. For example, the second target SNR of the second photoacoustic signal corresponding to the target region and the second reference SNR of the second photoacoustic signal corresponding to the reference region may be determined by the following steps S2701 to S2705:
Step S2701: determining a second noise reference value based on the second photoacoustic signal;
As an example, the median of the second photoacoustic signal may be taken as the second noise reference value.

Step S2702: determining an average of non-zero signal intensities in the second photoacoustic signal corresponding to the target region, so as to obtain a third effective signal intensity;
Step S2703: obtaining the second target SNR based on a ratio of the third effective signal intensity to the second noise reference value;

It should be noted that when there is no signal in the target region, the second target SNR is 0.

As an example, the second target SNR may be calculated by the following formula:

$$S N R 02 = db ( signal 03 / N )$$

where SNR02 represents the second target SNR, and signal03 represents the third effective signal intensity.

Step S2704: determining an average of non-zero signal intensities in the second photoacoustic signal corresponding to the reference region, so as to obtain a fourth effective signal intensity; and
Step S2705: obtaining the second reference SNR based on a ratio of the fourth effective signal intensity to the second noise reference value.

It should be noted that when there is no signal in the reference region, the second reference SNR is 0.

As an example, the second reference SNR may be calculated by the following formula:

$$SNR\_re2 = db ( signal 04 / N )$$

where SNR_re2 represents the second reference SNR, and signal04 represents the fourth effective signal intensity.

In this embodiment, the SNRs obtained based on the ratios of the effective signal intensities to the noise reference values are quantifiable, facilitating subsequent analysis and comparison.

In some embodiments, obtaining the first confidence level of the blood oxygen saturation signal in the target region based on the first target SNR, the first reference SNR, the second target SNR, and the second reference SNR may include:
Step S2801: determining a first ratio of the first target SNR to the first reference SNR, and a second ratio of the second target SNR to the second reference SNR; and
Step S2802: determining the first confidence level based on the sum of the first ratio and the second ratio, where the first confidence level is directly proportional to the sum of the first ratio and the second ratio.

Specifically, obtaining the confidence level of the blood oxygen saturation signal in the target region based on the first target SNR, the first reference SNR, the second target SNR, and the second reference SNR may also include: determining a depth coefficient corresponding to an imaging depth of the target region in photoacoustic imaging; and determining the first confidence level based on the depth coefficient, where the first confidence level is directly proportional to the depth coefficient.

It should be noted that the depth coefficient is adjustable within a range of 0 to 1. The coefficient can be dynamically adjusted in response to depth position changes in either the target region or the reference region.

As an example, since the intensities of the first and second photoacoustic signals significantly decrease with the increase of the depth of the imaging tissue, the depth coefficient Para2 may be set as follows:

$$depth < 0.25 cm or depth \geq 3 cm : Para 2 = 0.5 ;$$

$$0.25 \leq depth < 1 cm : Para 2 = 1 ;$$

$$1 \leq depth < 1.5 cm : Para 2 = 0.95 ;$$

$$1.5 \leq depth < 2 cm : Para 2 = 0.9 ;$$

$$2 \leq depth < 2.5 cm : Para 2 = 0.85 ;$$

$$2.5 \leq depth < 3 cm : Para 2 = 0.8 .$$

More specifically, obtaining the first confidence level of the blood oxygen saturation signal in the target region based on the first target SNR, the first reference SNR, the second target SNR, and the second reference SNR may further include: determining the first confidence level based on a reference coefficient, where the first confidence level is inversely proportional to the reference coefficient.

The reference coefficient is related to at least one of the following factors: the type of the target tissue, the energy level of the first laser pulse and the energy level of the second laser pulse.

It should be noted that the reference coefficient is adjustable withing a range of 0 to 1, and is used to determine the numerical relationship between the target SNR and the reference SNR. According to fundamental principles of photoacoustic imaging, the skin layer substantially absorbs light while being positioned superficially. Consequently, this layer typically generates stronger photoacoustic signals relative to subsurface blood flow signals. Given the positional stability of the skin layer, its photoacoustic signal intensity exhibits minimal spatial variation and thus serves as a reliable reference standard. However, it should be noted that significant variations in this parameter may occur due to different application scenarios, different objects under examination, and changes in laser output energy. By way of example, representative values derived from standard clinical practice include a reference coefficient ranging from 0.5 to 0.8 for metacarpal/forearm regions.

As an example, a normalized SNR coefficient may be obtained based on the sum of the first ratio and the second ratio, and the reference coefficient. For instance, the normalized SNR coefficient may be derived through the following formula:

$$Para 1 = ( \frac{S N R 01}{SNR\_re1} + \frac{S N R 02}{SNR\_re2} ) / ( 2 * default )$$

where Para1 represents the normalized SNR coefficient, and default represents the reference coefficient.

As another example, the first confidence level of the blood oxygen saturation signal in the target region may be calculated by the following formula:

$$confidence = Para 1 * Para 2$$

where confidence represents the first confidence level, and Para2 represents the depth coefficient.

Correspondingly, the first confidence level confidence may be recalculated repeatedly until the target region is changed (e.g. by a user) or the calculation of the first confidence level is stopped.

In this embodiment, the usage of the depth coefficient and the reference coefficient for the calculation of the first confidence level is conducive to improving the accuracy and reliability of the calculation result of the first confidence level.

In some embodiments, the calculation of the first confidence level may exclude the depth coefficient, for example, Para2 may be preset to 1.

In some embodiments, the reference region includes a skin layer region.

Specifically, when the reference region is a skin layer region, the determination of the reference region in the blood oxygen saturation image may include: subtracting the first photoacoustic signal or the second photoacoustic signal from the blood oxygen saturation signal to obtain a difference signal, and determining the reference region based on the difference signal.

As an example, since the skin layer is the first human tissue contacting light, with its physiological structure position being relatively fixed and located at a shallower depth in the image, the vast majority of photoacoustic imaging applications may traverse the skin layer imaging process. However, the skin layer reflects and absorbs a large amount of light, which appears as a very strong deep red signal in single-wavelength photoacoustic images, but is eliminated in blood oxygen saturation results due to the absence of blood flow signals. By utilizing this difference, the location of the skin layer can be determined. Thus, the original uploaded data image and the blood oxygen saturation image undergo processing, wherein the logical OR result of both images subtracts the blood oxygen saturation image result. The resultant data image then enables determination of the precise location of the skin layer region through a window traversal search method.

In this embodiment, by subtracting the first photoacoustic signal or the second photoacoustic signal from the blood oxygen saturation signal, a difference signal may be obtained, which can intuitively reflect the difference between the skin layer region and the blood oxygen saturation signal.

In some embodiments, determining the target region of the blood oxygen saturation image may include: determining the target region in response to a selection operation on the target region.

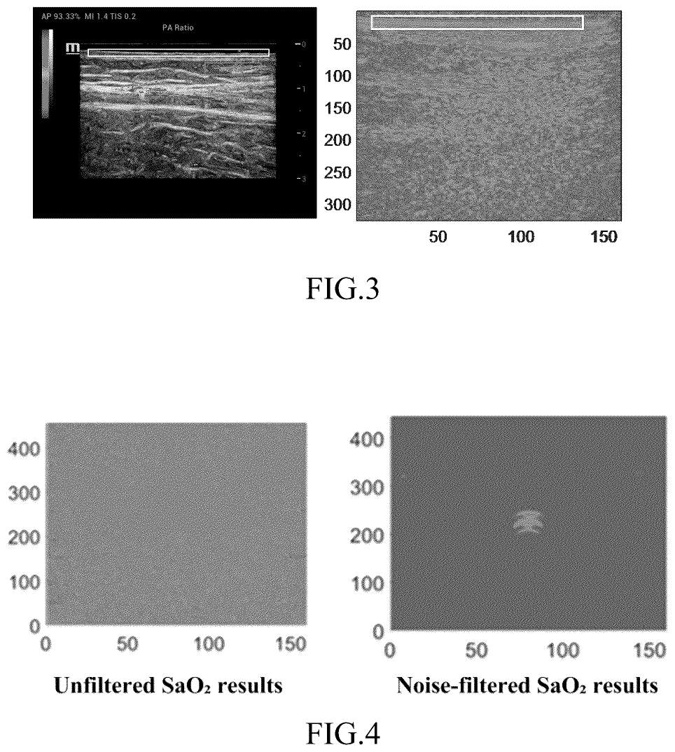

For example, as shown in FIG. 3, the coordinate parameters defining the target region selected manually by a user may be obtained. Using these coordinate parameters defines the region outlined by the red box in FIG. 3. The coordinate parameters defining the reference region (e.g., the skin layer region) may be determined using an automatic search algorithm. These coordinate parameters define the region indicated by the white selection box in FIG. 3 may be obtained.

The collected photoacoustic data contains significant noise, such that direct use thereof for blood oxygen saturation calculation results in substantial errors in the results; therefore, noise filtering of the data is required prior to data processing.

In some embodiments, before obtaining the blood oxygen saturation signal based on the first photoacoustic signal and the second photoacoustic signal, the photoacoustic imaging method may further include the following steps S2401 to S2404:
Step S2401: determining a first noise reference value based on the first photoacoustic signal;
Specifically, determining the first noise reference value based on the first photoacoustic signal includes: determining the first noise reference value based on an imaging depth of the target tissue in photoacoustic imaging.

Step S2402: determining a first noise range based on the first noise reference value, and filtering out the first photoacoustic signal within the first noise range;
Step S2403: determining a second noise reference value based on the second photoacoustic signal. Specifically, determining the second noise reference value based on the second photoacoustic signal includes: determining the second noise reference value based on the imaging depth of the target tissue in photoacoustic imaging.

Step S2404: determining a second noise range based on the second noise reference value, and filtering out the second photoacoustic signal within the second noise range.

As shown in FIG. 4, it can be seen that by filtering out the noise in the first and second photoacoustic signals, the interference of noise can be effectively removed.

In this embodiment, by performing a filtering operation on the first and second photoacoustic signals, noise can be effectively suppressed, the quality of the signal can be improved, and thus the accuracy of the blood oxygen saturation signal can be enhanced.

In some embodiments, determining the first noise reference value based on the imaging depth of the target tissue in photoacoustic imaging and the first photoacoustic signal includes:
Step S24011: when the imaging depth of the target tissue in photoacoustic imaging is less than or equal to a first preset depth, obtaining the first noise reference value based on the statistical value of photoacoustic signal intensities corresponding to the region within the first depth range in the first photoacoustic signal, where the first depth range corresponds to the surface layer of the tissue, including a range from a second preset depth within the first preset depth to the shallowest depth.

As an example, the statistical value may include the median or mode, etc.

Step S24012: when the imaging depth of the target tissue in photoacoustic imaging is greater than the first preset depth, obtaining the first noise reference value based on the statistical value of photoacoustic signal intensities corresponding to the region within the second depth range in the first photoacoustic signal, where the second depth range corresponds to the bottom layer of the tissue, including a range from a third preset depth within the first preset depth to the deepest depth.

As an example, when the reference region is the skin layer region, the first preset depth may be 3 cm.

The determination of the second noise reference value based on the imaging depth of the target tissue in photoacoustic imaging and the second photoacoustic signal may include:
Step S24031: when the imaging depth of the target tissue in photoacoustic imaging is less than or equal to the first preset depth, obtaining the second noise reference value based on the statistical value of photoacoustic signal intensities corresponding to a region within the first depth range in the second photoacoustic signal, where the first depth range corresponds to the surface layer of the tissue, including a range from the second preset depth within the first preset depth to the shallowest depth; and
Step S24032: when the imaging depth of the target tissue in photoacoustic imaging is greater than the first preset depth, obtaining the second noise reference value based on the statistical value of photoacoustic signal intensities corresponding to the region within the second depth range in the second photoacoustic signal, where the second depth range corresponds to the deep layer of the tissue, including a range from the third preset depth within the first preset depth to the deepest depth.

As an example, when the reference region is the skin layer region, the median is selected as the statistical value for filtering. Specifically, a reference noise signal range is selected based on the depth of the image (the first frame of image data collected). When the depth is less than or equal to 3 cm, the median of the signal intensities in the initial 5 mm region in the depth direction is considered as the first noise reference value N; and when the depth is greater than 3 cm, the median of the signal intensities in the final 5 mm region of the image in the depth direction is considered as the first noise reference value N.

As another example, a noise ratio parameter theta is provided, and the first noise range is determined based on theta and N. For instance, the first noise range comprises values less than N/theta. Values falling within the first noise range (i.e., less than N/theta) are regarded as noise and set to zero. Here, the noise ratio parameter theta is adjustable within a range of 0.05 to 0.1. As shown in FIG. 5, the aforementioned median filtering method filters out noise interference in the image.

In this embodiment, different depth ranges are adopted to determine the noise reference value based on the depth difference, which makes the determination of the noise reference value more depth-adaptive and enables better processing of signals at different depths.

In some embodiments, displaying the first confidence level includes: displaying a numerical value of the first confidence level; or, determining a confidence interval corresponding to the first confidence level and displaying a confidence identifier corresponding to the confidence interval. This intuitive display enables users to assess the reliability and accuracy of the displayed blood oxygen saturation measurement.

Specifically, the photoacoustic imaging method may further include: determining the first confidence level corresponding to multiple frames of blood oxygen saturation images, and simultaneously displaying a plurality of confidence identifiers according to the first confidence level corresponding to the multiple frames of blood oxygen saturation image.

As an example, the blood oxygen saturation may be calculated through the following formula:

$$S a O 2 = \frac{{C}_{HbO 2}}{{C}_{Hb} + {C}_{HbO 2}} = \frac{{ε}_{Hb}^{{λ}_{1}} {A}^{{λ}_{2}} - {ε}_{Hb}^{{λ}_{2}} {A}^{{λ}_{1}}}{{A}^{{λ}_{1}} ( {ε}_{HbO 2}^{{λ}_{2}} - {ε}_{Hb}^{{λ}_{2}} ) + {A}^{{λ}_{2}} ( {ε}_{Hb}^{{λ}_{1}} - {ε}_{HbO 2}^{{λ}_{1}} )} \times 100 \%$$

where C_{HbO2} represents the content of HbO2, C_{Hb} represents the content of Hb, λ₁ represents the first wavelength, λ₂ represents the second wavelength,

$${ε}_{Hb}^{{λ}_{1}}$$

represents the absorption coefficient of Hb at λ₁,

$${ε}_{Hb}^{{λ}_{2}}$$

represents the absorption coefficient of Hb at λ₂,

$${ε}_{HbO 2}^{{λ}_{1}}$$

represents the absorption coefficient of HbO2 at λ₁,

$${ε}_{HbO 2}^{{λ}_{2}}$$

represents the absorption coefficient of HbO2 at λ₂, A^{λ}^{₁} represents the intensity information of the photoacoustic signal obtained at λ₁, and A^{λ}^{₂} represents the intensity information of the photoacoustic signal obtained at λ₂.

It should be noted that during the calculation of blood oxygen saturation, any measurement values less than or equal to zero, which may result from noise, are assigned values (HbO(HbO<=0)=1e−5; Hb_total(Hb_total<=0)=1e−3;) to effectively exclude such invalid values from the SaO2 calculation result, and signals below a threshold of 0.4 are filtered out.

As another example, the first wavelength may be 750 nm and the second wavelength may be 830 nm. Alternatively, the first and second wavelengths may also be other suitable wavelength values.

More specifically, the photoacoustic imaging method further includes: when displaying a target frame of blood oxygen saturation image among the multiple frames of blood oxygen saturation image, highlighting the confidence indicator corresponding to the target frame of blood oxygen saturation image.

More specifically, the photoacoustic imaging method further includes: when receiving a selection instruction for a target confidence indicator from a plurality of confidence indicators, displaying the blood oxygen saturation image corresponding to the target confidence indicator.

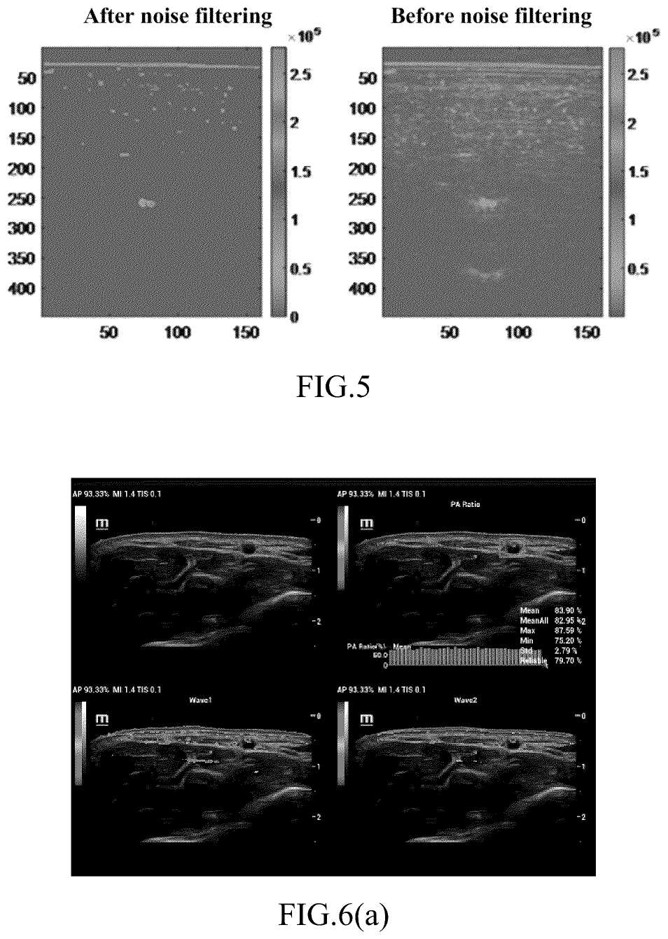

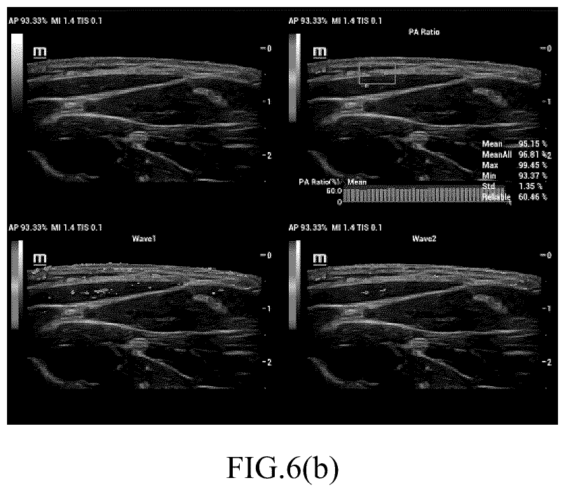

As an example, as shown in FIGS. 6(a) and 6(b), the results of confidence level can be dynamically displayed in the form of a histogram.

In this embodiment, by displaying the numerical value or confidence interval of the confidence level and the corresponding confidence indicator, the confidence level of different blood oxygen saturation images can be intuitively reflected, facilitating users to quickly understand and judge. By simultaneously displaying the plurality of confidence indicators, it is convenient for users to compare the confidence level of the multiple frames of blood oxygen saturation image, which is helpful for users to better evaluate and select.

Exemplarily, different color histogram results are output based on an interval in which the obtained value of the confidence level falls. For instance:
0.8≤Confidence<1 Display: High confidence level (green)
0.6≤Confidence<0.8 Display: Medium confidence level (yellow)
0.4≤Confidence<0.6 Display: Low confidence level (red)
0≤Confidence<0.4 Display: No confidence level (blue)

Using distinct colors to visually differentiate histogram results enables intuitive representation of the confidence levels associated with different blood oxygen saturation images, thereby enabling users to rapidly assess their validity.

In some embodiments, the photoacoustic imaging method further includes: establishing a correspondence between the first photoacoustic signal and the first laser pulse, and between the second photoacoustic signal and the second laser pulse, based on a signal acquisition timing sequence; or establishing a correspondence between the first photoacoustic signal and the first laser pulse, and between the second photoacoustic signal and the second laser pulse, based on signal amplitudes; wherein the first wavelength is greater than the second wavelength, and the signal amplitude of the second photoacoustic signal is greater than that of the first photoacoustic signal. The correspondence is used for determining, from the photoacoustic signals received by the transducer, the first photoacoustic signal corresponding to the first laser pulse and the second photoacoustic signal corresponding to the second laser pulse.

In this embodiment, establishing the correspondence based on the signal acquisition timing or on signal amplitude ensures the temporal or amplitude alignment of the first photoacoustic signal with the first laser pulse, and of the second photoacoustic signal with the second laser pulse. This enhances the accuracy and reliability of the blood oxygen saturation signal.

In some embodiments, the photoacoustic imaging method further includes: obtaining a blood oxygen saturation measurement result based on the blood oxygen saturation signal of the target region, and displaying the blood oxygen saturation measurement result on a display interface.

As an example, for each pixel, the deoxyhemoglobin content and the oxyhemoglobin content can be determined. This determination is based on: (1) the pixel value from the image data associated with the first photoacoustic signal and the pixel value from the image data associated with the second photoacoustic signal, and (2) the absorption coefficient of deoxyhemoglobin associated with the first laser pulse, the absorption coefficient of oxyhemoglobin associated with the first laser pulse, the absorption coefficient of deoxyhemoglobin associated with the second laser pulse, and the absorption coefficient of oxyhemoglobin associated with the second laser pulse. Based on the deoxyhemoglobin content and oxyhemoglobin content determined for each pixel, the blood oxygen saturation for each pixel is derived. Thereafter, the value of the blood oxygen saturation for each pixel can be assigned as the pixel value of that pixel, or the value of the blood oxygen saturation for each pixel can be calculated using a preset algorithm to obtain the pixel value of that pixel. Based on the pixel value of each pixel, a blood oxygen saturation image can be generated.

In some embodiments, the photoacoustic imaging method of the present application further includes: generating a quality control map of blood oxygen saturation for the target region based on the first confidence level, and displaying the quality control map of blood oxygen saturation, where different hues or different shades of the same hue in the quality control map of blood oxygen saturation are used to represent different levels of the first confidence level. For example, green represents high confidence level, yellow represents medium confidence level, and red represents low confidence level.

In this embodiment, by displaying the quality control map of blood oxygen saturation, the first confidence level of the blood oxygen saturation signal can be visually presented.

In some embodiments, the photoacoustic imaging method further includes: determining a first emission energy and the first wavelength of the first laser pulse, as well as a second emission energy and the second wavelength of the second laser pulse; obtaining pre-established correspondence relationships between different wavelengths, different emission energies, and blood oxygen saturation deviations; determining the blood oxygen saturation deviations corresponding to the first emission energy, the first wavelength, the second emission energy and the second wavelength according to the pre-established correspondence relationships, and determining the second confidence level of the blood oxygen saturation signal in the target region based on the blood oxygen saturation deviations; and determining the confidence level of the blood oxygen saturation signal in the target region based on the first confidence level and the second confidence level. It should be noted that, photoacoustic imaging of simulated biological tissues can be performed based on different wavelengths and different emission energies to obtain simulated measurement results of blood oxygen saturation corresponding to the different wavelengths and the different emission energies. These simulated measurement results of blood oxygen saturation are then compared with a blood oxygen saturation reference value (i.e., a theoretical reference value) to obtain a deviation of blood oxygen saturation. For example, photoacoustic imaging of simulated biological tissue is performed using the above-mentioned first emission energy and first wavelength, and/or the second emission energy and second wavelength to obtain simulated measurement results of blood oxygen saturation corresponding to the first emission energy and first wavelength, and/or the second emission energy and second wavelength. These simulated measurement results of blood oxygen saturation are then compared with the blood oxygen saturation reference value to obtain a deviation of blood oxygen saturation corresponding to the first emission energy and first wavelength, and/or the second emission energy and second wavelength.

In this embodiment, by adding the assessment of the second confidence level, the measurement accuracy can be improved.

In some embodiments, the photoacoustic imaging method further includes: determining a vascular depth in the target region and a vascular orientation in the target region; obtaining pre-established correspondence relationships between different vascular depths, different vascular orientations and blood oxygen saturation deviations; determining the blood oxygen saturation deviation corresponding to the vascular depth and the vascular orientation according to the pre-established correspondence relationships, and determining the third confidence level of the blood oxygen saturation signal in the target region based on said blood oxygen saturation deviation; and determining the confidence level of the blood oxygen saturation signal in the target region based on the first confidence level and the third confidence level. It should be noted that photoacoustic imaging of simulated biological tissue can be performed based on different vascular depths and different vascular orientations to obtain simulated blood oxygen saturation measurements corresponding to the different vascular depths and the different vascular orientations, and the simulated blood oxygen saturation measurements are compared with a blood oxygen saturation reference value (i.e., a theoretical reference value) to obtain a corresponding blood oxygen saturation deviation.

In this embodiment, the influence of vascular orientation and blood flow depth on the confidence level measurement result can be fully considered, thereby improving the measurement accuracy.

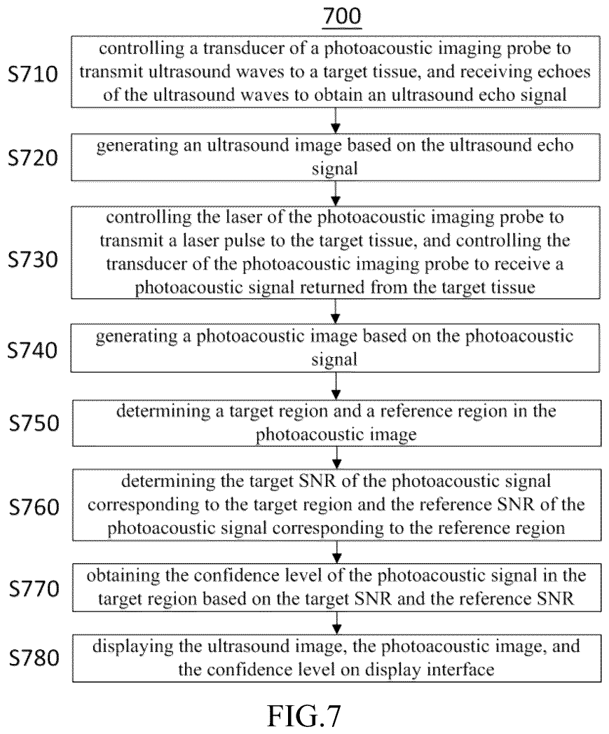

Below, a photoacoustic imaging method according to another embodiment of the present disclosure will be described with reference to FIG. 7. FIG. 7 is a schematic flowchart of a photoacoustic imaging method 700 according to an embodiment of the present disclosure.

As shown in FIG. 7, the photoacoustic imaging method 700 according to an embodiment of the present disclosure includes the following steps:
Step S710: controlling the transducer of a photoacoustic imaging probe to transmit ultrasound waves to a target tissue, and receive echoes of the ultrasound waves to obtain an ultrasound echo signal;
Step S720: generating an ultrasound image based on the ultrasound echo signal;
Step S730: controlling the laser of the photoacoustic imaging probe to transmit a laser pulse to the target tissue, and controlling the transducer of the photoacoustic imaging probe to receive a photoacoustic signal returned from the target tissue;
Step S740: generating a photoacoustic image based on the photoacoustic signal;
Step S750: determining a target region and a reference region in the photoacoustic image;
Step S760: determining the target SNR of the photoacoustic signal corresponding to the target region and the reference SNR of the photoacoustic signal corresponding to the reference region;
Step S770: obtaining the confidence level of the photoacoustic signal in the target region based on the target SNR and the reference SNR; and
Step S780: displaying the ultrasound image, the photoacoustic image, and the confidence level on display interface.

The photoacoustic imaging method 700 of this disclosed embodiment differs primarily from the photoacoustic imaging method 200 described above in the following aspects: first, the number of laser pulses is not limited to one (multiple pulses may be used); secondly, the photoacoustic image generated from the photoacoustic signal are displayed concurrently with the ultrasound image and the confidence level on the display interface. Further implementation details for the method 700 may be found in the corresponding description of the method 200 and are not repeated herein.

According to the photoacoustic imaging method 700 of the present embodiment, the confidence level of the photoacoustic signal in the target region is obtained based on the target SNR and the reference SNR, thereby improving reliability and effectiveness of the confidence level display result. The method concurrently displays the photoacoustic image, the ultrasound image and the confidence level on the display interface, facilitating analysis and processing of the displayed results.

In some embodiments, determining the target SNR of the photoacoustic signal corresponding to the target region and the reference SNR of the photoacoustic signal corresponding to the reference region includes:
Step S7501: determining a noise reference value based on the imaging depth of the target tissue in photoacoustic imaging and the photoacoustic signal;
Step S7502: determining the average of non-zero signal intensities in the photoacoustic signal corresponding to the target region to obtain the first effective signal intensity;
Step S7503: obtaining the target SNR based on the ratio of the first effective signal intensity to the noise reference value;
Step S7504: determining the average of non-zero signal intensities in the photoacoustic signal corresponding to the reference region to obtain the second effective signal intensity; and
Step S7505: obtaining the reference SNR based on the ratio of the second effective signal intensity to the noise reference value.

Specifically, obtaining the confidence level of the photoacoustic signal in the target region based on the target SNR and the reference SNR includes: determining the ratio of the target SNR to the reference SNR, and determining the confidence level based on said ratio, where the confidence level is directly proportional to said ratio.

More specifically, obtaining the confidence level of the photoacoustic signals in the target region based on the target SNR and the reference SNR also includes: determining the depth coefficient corresponding to the imaging depth of the target region in photoacoustic imaging; and determining the confidence level based on the depth coefficient, where the confidence level is directly proportional to the depth coefficient.

More specifically, obtaining the confidence level of the photoacoustic signals in the target region based on the target SNR and the reference SNR also includes: determining the confidence level based on the reference coefficient, where the confidence level is inversely proportional to the reference coefficient.

The reference coefficient correlated with at least one of the following factors: the type of the target tissue, and the energy level of the laser pulse.

In this embodiment, using the depth coefficient and the reference coefficient for the calculation of confidence level is conducive to improving the accuracy and reliability of the confidence level calculation results.

In some embodiments, the reference region includes a skin layer region.

As an example, since the skin layer is the first human tissue to come into contact with light with a relatively fixed physiological structure position at a shallower imaging depth, most photoacoustic imaging applications require imaging through the skin layer. The skin layer both reflects and absorbs a large amount of light, which appears as intensely strong deep red signals in single-wavelength photoacoustic images, thus enabling precise localization of the skin layer.

In some embodiments, before determining the target region and the reference region in the photoacoustic image, the photoacoustic imaging method further includes:
Step S7401: determining a noise reference value based on the imaging depth of the target tissue in photoacoustic imaging and the photoacoustic signal; and
Step S7402: determining a noise range based on the noise reference value, and filtering out the photoacoustic signals within the noise range.

In this embodiment, by performing a filtering operation on the photoacoustic signals, noise can be effectively suppressed and the quality of the signals can be improved.

In some embodiments, displaying the confidence level includes: displaying the numerical value of the confidence level; or, determining the confidence interval corresponding to the confidence level and displaying the confidence identifier corresponding to the confidence interval.

Specifically, the photoacoustic imaging method further includes: determining the confidence level corresponding to multiple frames of photoacoustic images and simultaneously displaying multiple confidence identifiers according to the confidence level associated with the multiple frames of photoacoustic images.

In this embodiment, by displaying the numerical value or confidence interval of the confidence level and the corresponding confidence identifier, the confidence level of different blood oxygen saturation images can be intuitively reflected, facilitating users to quickly understand and judge.

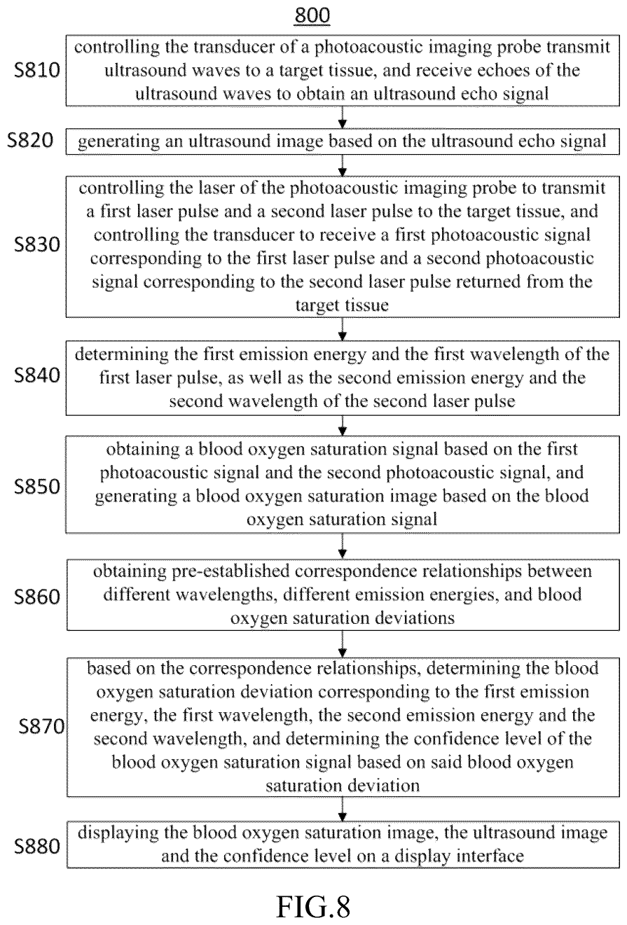

Next, the photoacoustic imaging method according to another embodiment of the present disclosure will be described with reference to FIG. 8. FIG. 8 is a schematic flowchart of the photoacoustic imaging method 800 according to an embodiment of the present disclosure.

As shown in FIG. 8, the photoacoustic imaging method 800 according to an embodiment of the present disclosure includes the following steps:
Step S810: controlling the transducer of the photoacoustic imaging probe transmit ultrasound waves to the target tissue, and receive echoes of the ultrasound waves to obtain an ultrasound echo signal;
Step S820: generating an ultrasound image based on the ultrasound echo signal;
Step S830: controlling the laser of the photoacoustic imaging probe to transmit a first laser pulse and a second laser pulse to the target tissue, and controlling the transducer to receive a first photoacoustic signal corresponding to the first laser pulse and a second photoacoustic signal corresponding to the second laser pulse that are returned from the target tissue;
Step S840: determining the first emission energy and the first wavelength of the first laser pulse, as well as the second emission energy and the second wavelength of the second laser pulse;
Step S850: obtaining a blood oxygen saturation signal based on the first photoacoustic signal and the second photoacoustic signal, and generating a blood oxygen saturation image based on the blood oxygen saturation signal;
Step S860: obtaining pre-established correspondence relationships between different wavelengths, different emission energies, and blood oxygen saturation deviations;
Step S870: based on the pre-established correspondence relationships, determining the blood oxygen saturation deviation corresponding to the first emission energy, the first wavelength, the second emission energy and the second wavelength, and determining the confidence level of the blood oxygen saturation signal based on said blood oxygen saturation deviation; and
Step S880: displaying the blood oxygen saturation image, the ultrasound image and the confidence level on a display interface.

The photoacoustic imaging method 800 of this disclosed embodiment differs primarily from the photoacoustic imaging method 200 described above in the following aspects: the blood oxygen saturation deviation corresponding to the first emission energy, the first wavelength, the second emission energy and the second wavelength is determined by obtaining pre-established correspondence relationships between different wavelengths, different emission energies, and blood oxygen saturation deviations, and the confidence level of the blood oxygen saturation signal in the target region is determined based on the blood oxygen saturation deviation. More specific details of the photoacoustic imaging method 800 of the present embodiment can be referred to the relevant description of the photoacoustic imaging method 200, and are not repeated herein.

According to the photoacoustic imaging method 800 of the present embodiment, by determining the blood oxygen saturation deviation corresponding to the first emission energy, the first wavelength, the second emission energy and the second wavelength based on the pre-established correspondence relationships between different wavelengths, different emission energies and the blood oxygen saturation deviations, and determining the confidence level of the blood oxygen saturation signal in the target region based on said blood oxygen saturation deviation, the reliability and effectiveness of the blood oxygen saturation display result can be improved. Meanwhile, the blood oxygen saturation image, the ultrasound image and the confidence level are displayed on the display interface, which is convenient for analyzing and processing the displayed results.

In some embodiments, the step of obtaining pre-established correspondence relationships between different wavelengths, different emission energies and blood oxygen saturation deviations includes:
Step S8601: performing simulated photoacoustic imaging on a simulated biological tissue at different wavelengths and different emission energies to obtain simulated blood oxygen saturation measurements corresponding to the different wavelengths and the different emission energies; and
Step S8602: obtaining blood oxygen saturation deviations corresponding to the different wavelengths and the different emission energies based on the deviations between the simulated blood oxygen saturation measurements and a blood oxygen saturation reference value.

In some embodiments, determining the first emission energy of the first laser pulse and the second emission energy of the second laser pulse includes: splitting the first laser pulse to obtain a first split laser beam, measuring the energy of the first split laser beam with an energy meter to obtain the first emission energy, and splitting the second laser pulse to obtain the second split laser beam, and measuring the energy of the second split laser beam with an energy meter to obtain the second emission energy.

In some embodiments, determining the first wavelength of the first laser pulse and the second wavelength of the second laser pulse includes: splitting the first laser pulse to obtain a third split laser beam, measuring the first wavelength of the third split laser beam with a spectrometer, and splitting the second laser pulse to obtain a fourth split laser beam, and measuring the second wavelength of the fourth split laser beam with a spectrometer.

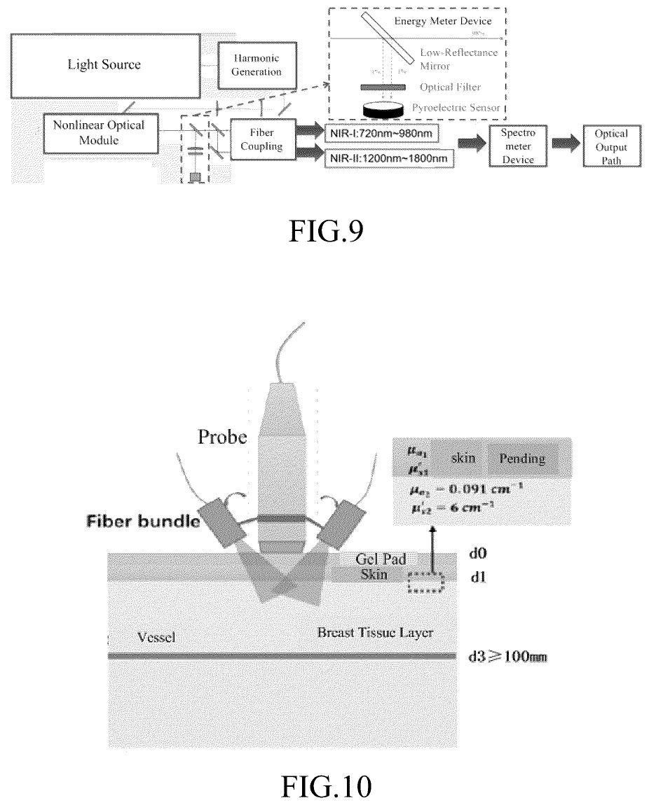

For example, as shown in FIG. 9, an internal energy meter is incorporated into the optical path. Prior to the fiber coupling section, the original transmitted laser A0 is split into the main path laser A1 and the branch path laser A2 by a low-reflectivity mirror. The main path laser A1 proceeds along the primary optical path to enter fiber coupling, while the branch path laser A2 is received by the internal energy meter to measure the actual transmitted energy. Similarly, an internal spectrometer is incorporated after the fiber coupling section. Using a similar method to split the light, the spectrometer collects a branch beam/split-off to measure the actual transmitted wavelength.

The method of modeling and simulation is adopted to simulate the specific influence of energy and wavelength deviations on the blood oxygen saturation results under different deviation conditions. The simulation can be based on any suitable modeling and simulation tool, such as COMSOL. COMSOL is a modeling and simulation tool based on the finite element method for analyzing static or dynamic physical objects or physical systems. It simulates actual physical phenomena by solving partial differential equation systems through mathematical methods. In this disclosure, COMSOL can be used to simulate the intensity/amplitude of photoacoustic signals generated after laser irradiation of biological tissues during photoacoustic imaging. By changing the preset energy and wavelength parameters, the errors introduced by deviations present in actual imaging

The following presents the simulation procedure using a simplified photoacoustic imaging model of breast tissue as an illustrative example.

First, confirm the model structure. Two optical fiber heads are mounted on both sides of the ultrasound probe, and the laser source is incident at a certain angle relative to both the probe plane and the vertical direction through two optical fibers. The target photoacoustic imaging sample can be divided into three layers: (1) Gel pad layer. A colorless, transparent, and sound-conductive material with an absorption coefficient μₐ and reduced scattering coefficient μₛ′ of 0. (2) Skin layer. (3) Breast layer. This is the target imaging region, containing blood vessels and having a thickness exceeding the maximum detectable depth of current photoacoustic imaging depth (5 cm). The absorption coefficient μₐ and reduced scattering coefficient μₛ′ of (2) and (3) are known, and all the above layers are considered as homogeneous media, as shown in FIG. 10.

Then, establish a forward model using the scattering equation(s) to describe the statistical transport behavior of the photon density wave in highly scattering media. For homogeneous media, the photon scattering model is equivalent to the Helmholtz equation. After the equations are implemented and solved in COMSOL, perform meshing and computation to obtain the photon fluence distribution (referred to here as light energy density). Since the intensity of the ultrasound signal generated at each location within biological tissue is proportional to the light energy absorbed by the tissue at that location, and this absorbed light energy is determined by the absorption coefficient and the photon fluence (light energy density) at that point. On this basis, the model is used with varying preset input energy and wavelength. This allows for generating different combinations of light energy density and absorption coefficient at the blood vessel location, thereby enabling the calculation of the corresponding blood oxygen saturation.

Finally, SaO2₁, SaO2₂, SaO2₃ . . . under different deviation conditions are calculated, and then compared with a theoretical SaO2 value (obtained under correct and stable energy and wavelength), and the confidence interval is determined based on the first deviation degree (Deviation1). It can be presented as follow, for example:
Deviation1<3% Display: High confidence level (green);
3%≤Deviation1<5% Display: Medium confidence level (yellow);
5%≤Deviation1<10% Display: Low confidence level (red);
10%≤Deviation1 Display: No confidence level (blue).

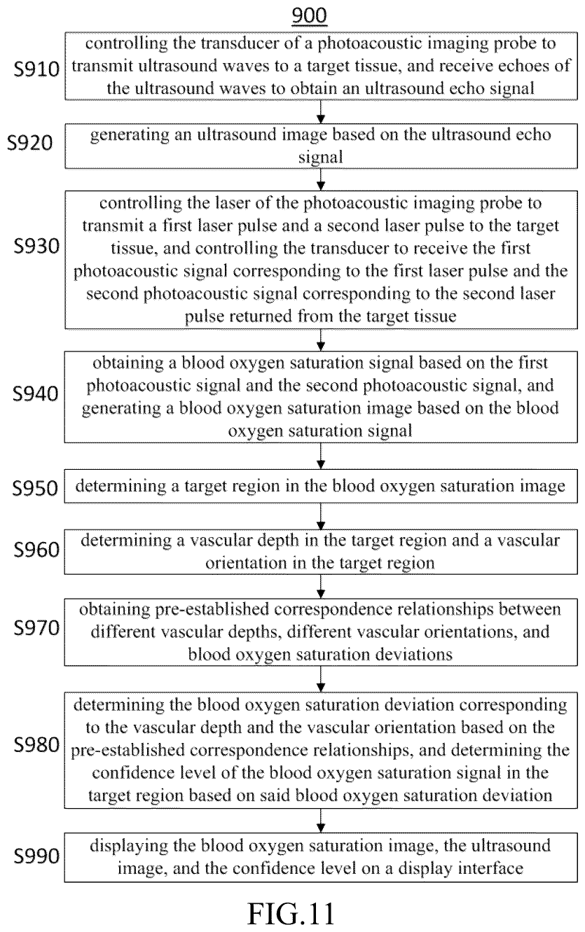

Below, the photoacoustic imaging method according to another embodiment of the present disclosure will be described with reference to FIG. 11. FIG. 11 is an exemplary schematic flowchart of the photoacoustic imaging method 900 according to an embodiment of the present disclosure.

As shown in FIG. 11, the photoacoustic imaging method 900 according to an embodiment of the present disclosure includes the following steps:
Step S910: controlling the transducer of a photoacoustic imaging probe to transmit ultrasound waves to a target tissue, and receive echoes of the ultrasound waves to obtain an ultrasound echo signal;
Step S920: generating an ultrasound image based on the ultrasound echo signal;
Step S930: controlling the laser of the photoacoustic imaging probe to transmit a first laser pulse and a second laser pulse to the target tissue, and controlling the transducer to receive the first photoacoustic signal corresponding to the first laser pulse and the second photoacoustic signal corresponding to the second laser pulse that are returned from the target tissue;
Step S940: obtaining a blood oxygen saturation signal based on the first photoacoustic signal and the second photoacoustic signal, and generating a blood oxygen saturation image based on the blood oxygen saturation signal;
Step S950: determining a target region in the blood oxygen saturation image;
Step S960: determining a vascular depth in the target region and a vascular orientation in the target region;
Step S970: obtaining pre-established correspondence relationships between different vascular depths, different vascular orientations, and blood oxygen saturation deviations;
Step S980: determining the blood oxygen saturation deviation corresponding to the vascular depth and the vascular orientation based on the pre-established correspondence relationships, and determining the confidence level of the blood oxygen saturation signal in the target region based on said blood oxygen saturation deviation; and
Step S990: displaying the blood oxygen saturation image, the ultrasound image, and the confidence level on a display interface.

The photoacoustic imaging method 900 of the present disclosed embodiment primarily differs from the photoacoustic imaging method 800 described above in the following aspects: the method 900 obtains the pre-established correspondence relationships between different vascular depths, different vascular orientations and blood oxygen saturation deviations, determines the blood oxygen saturation deviation corresponding to the vascular depth and vascular direction based on this pre-established correspondence, and determines the confidence level of the blood oxygen saturation signal in the target region based on the blood oxygen saturation deviation. More specific details of the photoacoustic imaging method 900 of the embodiment of the present disclosure can be referred to the relevant descriptions of the photoacoustic imaging methods 200 and 800, and are not repeated herein.

According to the photoacoustic imaging method 900 of the embodiment of the present disclosure, by determining the blood oxygen saturation deviation corresponding to the vascular depth and vascular orientation based on the pre-established correspondence relationships between different vascular depths, different vascular orientations and blood oxygen saturation deviations, and then determining the confidence level of the blood oxygen saturation signal in the target region based on the blood oxygen saturation deviation, the reliability and effectiveness of the displayed results of the blood oxygen saturation can be improved. Meanwhile, by displaying the blood oxygen saturation image, the ultrasound image and the confidence level on the display interface, it is convenient to analyze and process the displayed results.

In some embodiments, the step of obtaining pre-established correspondence relationships between different vascular depths, different vascular orientations and blood oxygen saturation deviations includes:
Step S9601: performing simulated photoacoustic imaging on simulated biological tissues with different vascular depths and orientations to obtain simulated blood oxygen saturation measurements corresponding to different vascular depths and orientations; and
Step S9602: obtaining the blood oxygen saturation deviations corresponding to different vascular depths and orientations, based on deviations between the simulated blood oxygen saturation measurements and a blood oxygen saturation reference value.

As an example, the same simulation modeling approach is adopted for comparative analysis, with the COMSOL tool retained as the simulation platform.

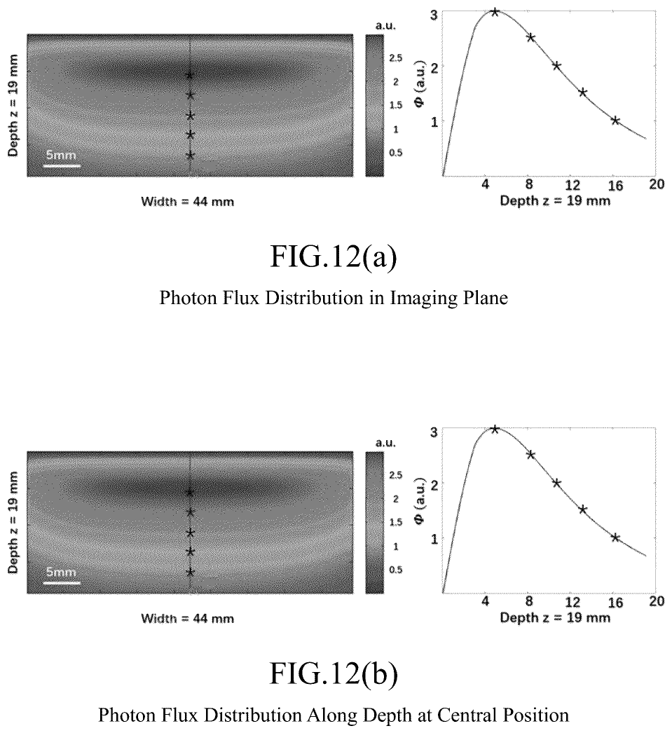

The modeling process is omitted. A breast model similar to that in the photoacoustic imaging method 800 is established, without initial vascular configuration. In a homogeneous medium, as shown in FIG. 12, the simulated photon fluence distribution in the breast model.

The depth of the blood vessels varies, resulting in different light energy densities, which in turn significantly affect the intensity of the generated photoacoustic signal, thereby influencing blood oxygen saturation calculations. During the simulation process, based on differences in light flux distribution, the blood vessel model is positioned and simulated at different depths incrementally, thereby clarifying the impact of depth on blood oxygen saturation calculations.

Further, in reality, the vascular orientation is often not completely perpendicular to the probe direction but forms a certain angle with the probe direction within the photoacoustic imaging plane. The impact of different vascular orientations on the calculation result of blood oxygen saturation can also be simulated via modeling. Specifically, a series of blood vessel models with angles of 15°, 30° . . . 90° relative to the probe direction can be set to clarify the influence of vascular orientation (angle) on the calculation result of blood oxygen saturation. It is worth noting that the vascular orientations may also form an angle with the photoacoustic imaging section, but here only the photoacoustic signal of blood flow projected onto the photoacoustic imaging section is concerned, so this angle has little impact on the result.

In photoacoustic images, the vascular depth is determined by the coordinate position of the blood flow photoacoustic signal within the ROI, and the contour envelope is recorded. The angle between the blood vessel and the probe orientation is determined by the morphological method of image processing. By combining different conditions of the depth position of the blood vessel and the angle with the probe orientation, the calculated SaO2₁, SaO2₂, SaO2₃ . . . are compared with the theoretical value of blood oxygen saturation. The confidence interval is defined based on the second deviation degree (Deviation2).
Deviation2<3% Display: High confidence level (green);
3%≤Deviation2<5% Display: Medium confidence level (yellow);
5%≤Deviation2<10% Display: Low confidence level (red);
10%≤Deviation2 Display: No confidence level (blue).

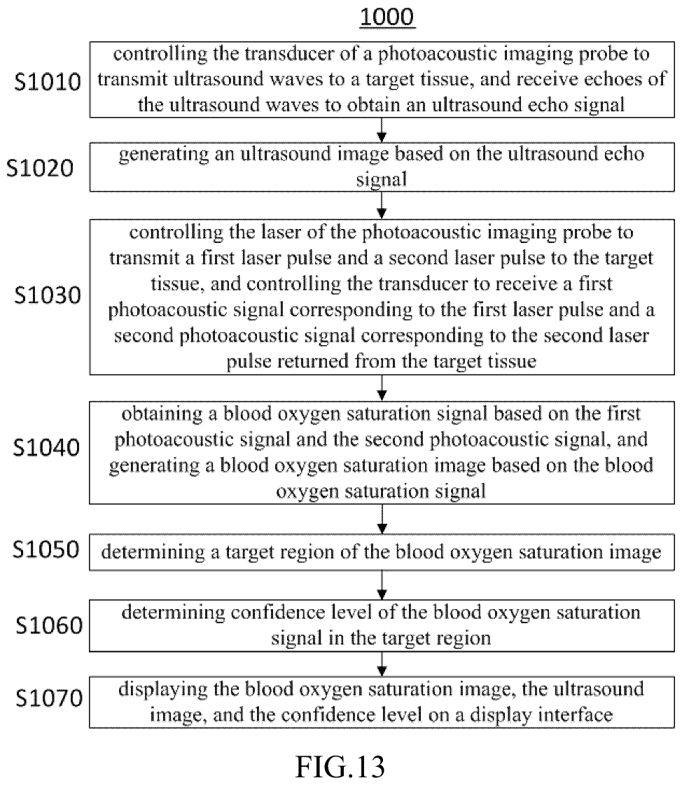

Below, the photoacoustic imaging method according to another embodiment of the present disclosure will be described with reference to FIG. 13. FIG. 13 is a schematic flowchart of the photoacoustic imaging method 1000 according to an embodiment of the present disclosure.

As shown in FIG. 13, the photoacoustic imaging method 1000 according to an embodiment of the present disclosure includes the following steps:
Step S1010: controlling the transducer of a photoacoustic imaging probe to transmit ultrasound waves to a target tissue, and receive echoes of the ultrasound waves to obtain an ultrasound echo signal;
Step S1020: generating an ultrasound image based on the ultrasound echo signal;
Step S1030: controlling the laser of the photoacoustic imaging probe to transmit a first laser pulse and a second laser pulse to the target tissue, and controlling the transducer to receive a first photoacoustic signal corresponding to the first laser pulse and a second photoacoustic signal corresponding to the second laser pulse that are returned from the target tissue;
Step S1040: obtaining a blood oxygen saturation signal based on the first photoacoustic signal and the second photoacoustic signal, and generating a blood oxygen saturation image based on the blood oxygen saturation signal;
Step S1050: determining a target region of the blood oxygen saturation image;
Step S1060: determining confidence level of the blood oxygen saturation signal in the target region; and
Step S1070: displaying the blood oxygen saturation image, the ultrasound image, and the confidence level on a display interface.

The main difference between the photoacoustic imaging method 1000 of the embodiment of the present disclosure and the photoacoustic imaging method 200 described above lies in that the method 1000 does not require the determination of a reference region for the blood oxygen saturation image. More specific details of the photoacoustic imaging method 200 of the embodiment of the present disclosure can be referred to the relevant description of the photoacoustic imaging method 200, and are not repeated here.

According to the photoacoustic imaging method 200 of the embodiment of the present disclosure, by displaying the blood oxygen saturation image, the ultrasound image and the confidence level on the display interface, it is convenient to analyze and process the displayed results.

In some embodiments, determining the confidence level of the blood oxygen saturation signal of the target region includes: obtaining at least two confidence parameters of the blood oxygen saturation signal of the target region according to at least two different confidence evaluation standards; and determining the confidence level of the blood oxygen saturation signal in the target region based on the at least two confidence parameters.

A photoacoustic imaging system is also provided in an embodiment of the present disclosure for implementing the above-mentioned photoacoustic imaging method 200 or photoacoustic imaging method 800. Referring again to FIG. 1, the photoacoustic imaging system may be implemented as the photoacoustic imaging system 100 shown in FIG. 1. The photoacoustic imaging system 100 may include a laser 110, a photoacoustic imaging probe 112, a transmit circuit 114, a receive circuit 116, a processor 118, and a display 120. Optionally, the photoacoustic imaging system 100 may also include a transmit/receive selection switch 122 and a beamforming module 124. The transmit circuit 114 and the receive circuit 116 may be connected to the photoacoustic imaging probe 112 through the transmit/receive selection switch 122. The laser 110 is configured to transmit laser to an imaging target, causing the imaging target to return photoacoustic signals. The receive circuit 116 is configured to control the photoacoustic imaging probe 112 to receive photoacoustic signals. The processor 118 is configured to execute the above-mentioned photoacoustic imaging method 200 or photoacoustic imaging method 800 to generate photoacoustic images based on the photoacoustic signals. The display 120 is configured to display the photoacoustic images. Additionally, the laser 110 may also be included inside the photoacoustic imaging probe 112 and be a component of the photoacoustic imaging probe 112. The relevant descriptions of each component may be referred to the above descriptions and are not repeated here.

The above only describes the main functions of each component of the photoacoustic imaging system. For more details, please refer to the relevant descriptions of the photoacoustic imaging methods 200, 700, 800, 900 and 1000. The photoacoustic imaging system 100 of the embodiments of the present disclosure compensates the photoacoustic image data in a targeted manner based on the light flux distribution result corresponding to the biological tissue information of the imaging target, thereby enhancing the photoacoustic signal intensities, increasing the imaging depth, and optimizing the quality and accuracy of the photoacoustic image.

Although the example embodiments have been described with reference to the accompanying drawings, it should be understood that the above example embodiments are merely illustrative and are not intended to limit the scope of the present disclosure. Those skilled in the art can make various changes and modifications without departing from the scope and spirit of the present disclosure. All these changes and modifications are intended to be included within the scope of the present disclosure as defined by the appended claims.

Those skilled in the art can appreciate that the units and algorithm steps described in the various examples in this disclosure can be implemented in electronic hardware, or in software and electronic hardware. The choice of whether to implement the functions in hardware or software depends on the specific application and design constraints of the technical solution. Skilled professionals can use different methods to implement the described functions for each specific application, but such implementation should not be considered as going beyond the scope of the present disclosure.

In several embodiments provided by the present disclosure, it should be understood that the disclosed devices and methods can be implemented in other ways. For example, the device embodiments described above are merely illustrative. For instance, the division of the units is merely a logical functional division. In actual implementation, there can be other division methods. For example, multiple units or components can be combined or integrated into another device, or some features can be ignored or not executed.

In the specification provided herein, a large number of specific details are described. However, it is to be understood that the embodiments of the present disclosure can be practiced without these specific details. In some instances, well-known methods, structures and techniques have not been shown in detail so as not to obscure the understanding of the specification.

Similarly, it should be understood that, for the sake of simplicity and to aid in understanding one or more aspects of the invention, various features of the invention may be grouped together in a single embodiment, figure, or description of the embodiment. However, the disclosure should not be construed as requiring more features than those explicitly recited in each claim. Rather, as reflected in the corresponding claims, the inventive concept lies in solving the corresponding technical problem with fewer features than those of a single disclosed embodiment. Therefore, the claims are hereby incorporated by reference into the specific embodiments, with each claim itself serving as a separate embodiment of the disclosure.

It will be understood by those skilled in the art that, except where the features are mutually exclusive, any combination of the features disclosed in this specification (including the accompanying claims, abstract and drawings), as well as all processes or units of any method or device disclosed herein, may be combined. Unless otherwise expressly stated, each feature disclosed in this specification (including the accompanying claims, abstract and drawings) may be replaced by an alternative feature which provides the same, equivalent or similar purpose.

Furthermore, it is understood by those skilled in the art that although some of the embodiments described herein include certain features but not others included in other embodiments, the combination of features from different embodiments is within the scope of the invention and forms different embodiments. For example, in the claims, any one of the embodiments claimed for protection can be used in any combination.

The various component embodiments of the present disclosure can be implemented in hardware, or in software modules running on one or more processors, or in a combination thereof. Those skilled in the art should understand that some or all of the functions of some modules according to the embodiments of the present disclosure can be implemented using a microprocessor or a digital signal processor (DSP) in practice. The present disclosure can also be implemented as a device program (e.g., a computer program and a computer program product) for performing part or all of the methods described herein. Such a program for implementing the present disclosure can be stored on a computer-readable medium or can have the form of one or more signals. Such signals can be downloaded from an Internet website, provided on a carrier signal, or provided in any other form.

It should be noted that the above examples are illustrative of the disclosure rather than restrictive, and that skilled persons in the art may design alternative embodiments without departing from the scope of the appended claims. In the claims, any reference symbols placed between parentheses should not be construed as limiting the claims. The disclosure can be implemented by means of hardware including several different components and by means of a suitably programmed computer. In a unitary claim listing several devices, several of these devices may be embodied by one and the same hardware item. The use of the terms “first”, “second”, “third”, etc. does not imply any order. These words should be interpreted as names.

The above description is merely a specific implementation of the present disclosure or an explanation of a specific implementation. The protection scope of the present disclosure is not limited to this. Those skilled in the art can easily think of variations or replacements within the technical scope disclosed by the present disclosure, and all such variations or replacements should be covered within the protection scope of the present disclosure. The protection scope of the present disclosure should be determined by the protection scope of the claims.

What is claimed is:

1. A photoacoustic imaging method, comprising:
controlling a transducer of a photoacoustic imaging probe to transmit ultrasound waves to a target tissue and receive echoes of the ultrasound waves to obtain an ultrasound echo signal;
generating an ultrasound image based on the ultrasound echo signal;
controlling a laser of the photoacoustic imaging probe to transmit a first laser pulse of a first wavelength and a second laser pulse of a second wavelength to the target tissue, and controlling the transducer of the photoacoustic imaging probe to receive a first photoacoustic signal corresponding to the first laser pulse and a second photoacoustic signal corresponding to the second laser pulse that are returned from the target tissue;
obtaining a blood oxygen saturation signal based on the first photoacoustic signal and the second photoacoustic signal, generating a blood oxygen saturation image based on the blood oxygen saturation signal, and determining a target region and a reference region in the blood oxygen saturation image;
determining a first target signal-to-noise ratio (SNR) of the first photoacoustic signal corresponding to the target region and a first reference SNR of the first photoacoustic signal corresponding to the reference region;
determining a second target SNR of the second photoacoustic signal corresponding to the target region and a second reference SNR of the second photoacoustic signal corresponding to the reference region;
obtaining a first confidence level of the blood oxygen saturation signal in the target region based on the first target SNR, the first reference SNR, the second target SNR, and the second reference SNR; and
displaying the blood oxygen saturation image, the ultrasound image, and the first confidence level on a display interface.

2. The photoacoustic imaging method according to claim 1, wherein determining a first target SNR of the first photoacoustic signal corresponding to the target region and a first reference SNR of the first photoacoustic signal corresponding to the reference region, comprises:
determining a first noise reference value based on the first photoacoustic signal;
determining an average of non-zero signal intensities in the first photoacoustic signal corresponding to the target region, so as to obtain a first effective signal intensity;
obtaining the first target SNR based on a ratio of the first effective signal intensity to the first noise reference value;
determining an average of non-zero signal intensities in the first photoacoustic signal corresponding to the reference region, so as to obtain a second effective signal intensity; and
obtaining the first reference SNR based on a ratio of the second effective signal intensity to the first noise reference value;
and wherein determining a second target SNR of the second photoacoustic signal corresponding to the target region and a second reference SNR of the second photoacoustic signal corresponding to the reference region, comprises:
determining a second noise reference value based on the second photoacoustic signal;
determining an average of non-zero signal intensities in the second photoacoustic signal corresponding to the target region, so as to obtain a third effective signal intensity;
obtaining the second target SNR based on a ratio of the third effective signal intensity to the second noise reference value;
determining an average of non-zero signal intensities in the second photoacoustic signal corresponding to the reference region, so as to obtain a fourth effective signal intensity; and
obtaining the second reference SNR based on a ratio of the fourth effective signal intensity to the second noise reference value.

3. The photoacoustic imaging method according to claim 1, wherein obtaining a first confidence level of the blood oxygen saturation signal in the target region based on the first target SNR, the first reference SNR, the second target SNR, and the second reference SNR, comprises:
determining a first ratio of the first target SNR to the first reference SNR and a second ratio of the second target SNR to the second reference SNR; and
determining the first confidence level based on a sum of the first ratio and the second ratio, wherein the first confidence level is positively correlated with the sum of the first ratio and the second ratio.

4. The photoacoustic imaging method according to claim 3, further comprising:
determining a depth coefficient corresponding to an imaging depth of the target region in photoacoustic imaging;
wherein determining the first confidence level based on a sum of the first ratio and the second ratio comprises:
determining the first confidence level based on the sum of the first ratio and the second ratio and the depth coefficient, wherein the first confidence level is also positively correlated with the depth coefficient.

5. The photoacoustic imaging method according to claim 3, further comprising:
obtaining a reference coefficient correlated with at least one of following factors: a type of the target tissue, an energy level of the first laser pulse, and an energy level of the second laser pulse;
wherein determining the first confidence level based on a sum of the first ratio and the second ratio comprises:
determining the first confidence level based on said sum of the first ratio and the second ratio and the reference coefficient, or
determining the first confidence level based on said sum of the first ratio and the second ratio, the depth coefficient, and the reference coefficient;
wherein the first confidence level is negatively correlated with the reference coefficient.

6. The photoacoustic imaging method according to claim 1, wherein the reference region includes a skin layer region.

7. The photoacoustic imaging method according to claim 6, wherein when the reference region is the skin layer region, determining a reference region in the blood oxygen saturation image comprises:
subtracting the first photoacoustic signal or the second photoacoustic signal from the blood oxygen saturation signal to obtain a difference signal; and
determining the reference region based on the difference signal.

8. The photoacoustic imaging method according to claim 1, wherein before obtaining a blood oxygen saturation signal based on the first photoacoustic signal and the second photoacoustic signal, the method further comprises:
determining a first noise reference value based on the first photoacoustic signal;
determining a first noise range according to the first noise reference value, and filtering out the first photoacoustic signal within the first noise range;
determining a second noise reference value based on the second photoacoustic signal; and
determining a second noise range according to the second noise reference value, and filtering out the second photoacoustic signal within the second noise range.

9. The photoacoustic imaging method according to claim 2, wherein determining a first noise reference value based on the first photoacoustic signal comprises: determining the first noise reference value based on an imaging depth of the target tissue in photoacoustic imaging and the first photoacoustic signal;
and wherein determining a second noise reference value based on the second photoacoustic signal comprises: determining the second noise reference value based on the imaging depth of the target tissue in photoacoustic imaging and the second photoacoustic signal.

10. The photoacoustic imaging method according to claim 9,
wherein determining the first noise reference value based on an imaging depth of the target tissue in photoacoustic imaging and the first photoacoustic signal, comprises:
when the imaging depth of the target tissue in photoacoustic imaging is less than or equal to a first preset depth, obtaining the first noise reference value based on a statistical value of photoacoustic signal intensities corresponding to regions within a first depth range in the first photoacoustic signal, wherein the first depth range extends from a second preset depth within the first preset depth to a shallowest depth; and
when the imaging depth of the target tissue in photoacoustic imaging is greater than the first preset depth, obtaining the first noise reference value based on a statistical value of photoacoustic signal intensities corresponding to regions within a second depth range in the first photoacoustic signal, wherein the second depth range extends from a third preset depth within the first preset depth to a deepest depth;
and wherein determining the second noise reference value based on the imaging depth of the target tissue in photoacoustic imaging and the second photoacoustic signal, comprises:
when the imaging depth of the target tissue in photoacoustic imaging is less than or equal to the first preset depth, obtaining the second noise reference value based on a statistical value of photoacoustic signal intensities corresponding to regions within the first depth range in the second photoacoustic signal, wherein the first depth range extends from the second preset depth within the first preset depth to the shallowest depth; and
when the imaging depth of the target tissue in photoacoustic imaging is greater than the first preset depth, obtaining the second noise reference value based on a statistical value of photoacoustic signal intensities corresponding to regions within the second depth range in the second photoacoustic signal, wherein the second depth range extends from the third preset depth within the first preset depth to the deepest depth.

11. The photoacoustic imaging method according to claim 1, wherein displaying the first confidence level comprises:
displaying a numerical value of the first confidence level;
or, determining a confidence interval corresponding to the first confidence level, and displaying a confidence indicator corresponding to said confidence interval.

12. The photoacoustic imaging method according to claim 11, further comprising:
determining first confidence levels corresponding to multiple frames of blood oxygen saturation image, and simultaneously displaying a plurality of confidence indicators based on the first confidence levels corresponding to the multiple frames of blood oxygen saturation image.

13. The photoacoustic imaging method according to claim 12, further comprising:
when displaying a target frame of blood oxygen saturation image among the multiple frames of blood oxygen saturation image, highlighting a confidence indicator corresponding to the target frame of blood oxygen saturation image.

14. The photoacoustic imaging method according to claim 12, further comprising:
when receiving a selection instruction for a target confidence indicator among the plurality of confidence indicators, displaying a blood oxygen saturation image corresponding to said target confidence indicator.

15. The photoacoustic imaging method according to claim 1, further comprising:
generating a quality control map of blood oxygen saturation for the target region based on the first confidence level, and displaying the quality control map of blood oxygen saturation, wherein different hues or different shades of a same hue in the quality control map of blood oxygen saturation are used to represent different levels of the first confidence level.

16. The photoacoustic imaging method according to claim 1, further comprising:
determining a first emission energy and the first wavelength of the first laser pulse, and a second emission energy and the second wavelength of the second laser pulse;
obtaining pre-established correspondence relationships between different wavelengths, different emission energies, and blood oxygen saturation deviations;
determining a blood oxygen saturation deviation corresponding to the first emission energy, the first wavelength, the second emission energy, and the second wavelength based on said correspondence relationships, and determining a second confidence level of the blood oxygen saturation signal in the target region according to said blood oxygen saturation deviation; and
determining an overall confidence level of the blood oxygen saturation signal in the target region based on the first confidence level and the second confidence level.

17. The photoacoustic imaging method according to claim 1, further comprising:
determining a vascular depth within the target region and a vascular orientation within the target region;
obtaining pre-established correspondence relationships between different vascular depths, different vascular orientations, and blood oxygen saturation deviations;
determining a blood oxygen saturation deviation corresponding to the vascular depth and vascular orientation based on said correspondence relationships, and determining a third confidence level of the blood oxygen saturation signal in the target region according to said blood oxygen saturation deviation; and
determining a confidence level of the blood oxygen saturation signal in the target region based on the first confidence level and the third confidence level.

18. A photoacoustic imaging method, comprising:
controlling a transducer of a photoacoustic imaging probe to transmit ultrasound waves to a target tissue and receive echoes of the ultrasound waves to obtain an ultrasound echo signal;
generating an ultrasound image based on the ultrasound echo signal;
controlling a laser of the photoacoustic imaging probe to transmit a laser pulse toward the target tissue, and controlling the transducer of the photoacoustic imaging probe to receive a photoacoustic signal returned from the target tissue;
generating a photoacoustic image based on the photoacoustic signal;
determining a target region and a reference region in the photoacoustic image;
determining a target signal-to-noise ratio (SNR) of the photoacoustic signal corresponding to the target region and a reference SNR of the photoacoustic signal corresponding to the reference region;
obtaining a confidence level of the photoacoustic signal in the target region based on the target SNR and the reference SNR; and
displaying the ultrasound image, the photoacoustic image, and the confidence level on a display interface.

19. The photoacoustic imaging method according to claim 18, wherein determining a target SNR of the photoacoustic signal corresponding to the target region and a reference SNR of the photoacoustic signal corresponding to the reference region, comprises:
determining a noise reference value based on an imaging depth of the target tissue in photoacoustic imaging and the photoacoustic signal;
determining an average of non-zero signal intensities in the photoacoustic signal corresponding to the target region, so as to obtain a first effective signal intensity;
obtaining the target SNR based on a ratio of the first effective signal intensity to the noise reference value;
determining an average of non-zero signal intensities in the photoacoustic signal corresponding to the reference region, so as to obtain a second effective signal intensity; and
obtaining the reference SNR based on a ratio of the second effective signal intensity to the noise reference value.

20. The photoacoustic imaging method according to claim 18, wherein displaying the confidence level comprises:
displaying a numerical value of the confidence level;
or, determining a confidence interval corresponding to the confidence level, and displaying a confidence indicator corresponding to the confidence interval.
